(19) United States
(12) Patent Application Publication
(10) Pub. No.: US 2025/0127534 A1
(43) Pub. Date: Apr. 24, 2025

(54) SURGICAL INSTRUMENT

(71) Applicant: LIVSMED INC., Seongnam-si (KR)

(72) Inventor: Hyun JOO, Seoul (KR)

(21) Appl. No.: 18/889,692

(22) Filed: Sep. 19, 2024

(30) Foreign Application Priority Data
Oct. 24, 2023 (KR) 10-2023-0143234

(51) Int. Cl. A61B 17/29; A61B 17/00

(52) U.S. Cl. CPC A61B 17/2909; A61B 2017/00367; A61B 2017/00477; A61B 2017/2901; A61B 2017/2925; A61B 2017/2927; A61B 2017/2929; A61B 2017/2939

(57) ABSTRACT

A surgical instrument includes an end tool including one or more jaws formed to be rotatable, and an end tool jaw pitch main pulley formed to be rotatable around an end tool pitch rotation shaft, a manipulation part configured to control a rotation of the end tool, a power transmission part including one or more jaw wires that are connected to the manipulation part to transmit a rotation of the manipulation part to the one or more jaws, and a connection part formed to extend in a first direction, having one end portion to which the end tool is coupled and another end portion to which the manipulation part is coupled to connect the end tool to the manipulation part, and including a bent part formed to be bent at least once while connecting the end tool to the manipulation part, wherein the manipulation part includes a pitch manipulation part including a manipulation part pitch main pulley formed to be rotatable around a pitch rotation shaft, and configured to control a pitch motion of the end tool, and a diameter of the manipulation part pitch main pulley is greater than a diameter of the end tool jaw pitch main pulley.

### CROSS-REFERENCE TO RELATED APPLICATION

This application is based on and claims priority under 35 USC § 119 to Korean Patent Application No. 10-2023-0143234, filed on Oct. 24, 2023, in the Korean Intellectual Property Office, the disclosure of which is incorporated by reference herein in its entirety.

### BACKGROUND

### 1. Field

The present disclosure relates to a surgical instrument, and more particularly, to a surgical instrument capable of being operated manually for use in laparoscopic surgery or various surgeries.

### 2. Description of the Related Art

Medically, surgery refers to the treatment of diseases by cutting, slitting, or manipulating the skin, mucous membranes, or other tissues using medical devices. In particular, open surgery in which the skin of the surgical site is incised and opened to treat, shape, remove organs or the like therein and the like cause problems such as bleeding, side effects, patient pain, scarring. Accordingly, recently, surgery performed by inserting only a medical device, for example, laparoscopic surgical instrument, microsurgical microscope, and the like by forming a predetermined hole in the skin or surgery using a robot has been spotlighted as an alternative.

A surgical instrument is a tool equipped with an end tool provided on one end of a shaft passing through a hole drilled in the skin, and is manipulated by a medical doctor by hand using a predetermined driving part or by a robot arm to perform surgery at the surgical site. The end tool provided on the surgical instrument performs a rotational motion, a gripping motion, a cutting motion, or the like through a predetermined structure.

However, in a conventional surgical instrument, due to the nature of the instrument, an arc-shaped frame, i.e. a bent part connecting a connection part to a manipulation part is formed elevated, which often causes interference between frames of a plurality of surgical instruments when used simultaneously.

The aforementioned background technology is technical information possessed by the inventor for derivation of the present disclosure or acquired by the inventor during the derivation of the present disclosure, and is not necessarily prior art disclosed to the public before the application of the present disclosure.

### SUMMARY

The present disclosure is directed to providing a surgical instrument capable of reducing the height of an arc-shaped frame to minimize interference between a plurality of instruments when used simultaneously while not affecting functionality.

Additional aspects will be set forth in part in the description which follows and, in part, will be apparent from the description, or may be learned by practice of the presented embodiments of the disclosure.

According to an aspect of the present disclosure, there is provided a surgical instrument including an end tool including one or more jaws formed to be rotatable, and an end tool jaw pitch main pulley formed to be rotatable around an end tool pitch rotation shaft, a manipulation part configured to control a rotation of the end tool, a power transmission part including one or more jaw wires that are connected to the manipulation part to transmit a rotation of the manipulation part to the one or more jaws, and a connection part formed to extend in a first direction (an X-axis), having one end portion to which the end tool is coupled and another end portion to which the manipulation part is coupled to connect the end tool to the manipulation part, and including a bent part formed to be bent at least once while connecting the end tool to the manipulation part, wherein the manipulation part includes a pitch manipulation part including a manipulation part pitch main pulley formed to be rotatable around a pitch rotation shaft, and configured to control a pitch motion of the end tool, and a diameter of the manipulation part pitch main pulley is greater than a diameter of the end tool jaw pitch main pulley.

In an embodiment of the present disclosure, the bent part may be bent on a plane perpendicular to the pitch rotation shaft of the pitch manipulation part.

In an embodiment of the present disclosure, a ratio of the diameter of the end tool jaw pitch main pulley to the diameter of the manipulation part pitch main pulley may range from 1:3.5 to 1:4.5.

In an embodiment of the present disclosure, the end tool jaw pitch main pulley may be connected to the manipulation part pitch main pulley through the one or more jaw wires, and a rotation angle of the end tool jaw pitch main pulley when the at least one or more jaw wires move may be greater than a rotation angle of the manipulation part pitch main pulley when the at least one or more jaw wires move.

In an embodiment of the present disclosure, a ratio of the rotation angle of the manipulation part pitch main pulley to the rotation angle of the end tool jaw pitch main pulley may range from 1:3.5 to 1:4.5.

In an embodiment of the present disclosure, the bent part may be formed not to make contact with the manipulation part but to be positioned maximally adjacent to a rotation radius of the manipulation part, when the manipulation part is controlled to pitch-rotate the end tool at a maximum rotation angle.

In an embodiment of the present disclosure, the end tool may include an end tool hub formed to internally accommodate at least some of the jaws, and an end tool pitch pulley formed on an end portion of the end tool hub at a proximal end side, the power transmission part may further include a pitch wire coupled to the end tool pitch pulley to rotate the end tool pitch pulley, the manipulation part may further include a manipulation part pitch wire pulley around which the pitch wire is wound, and a diameter of the manipulation part pitch wire pulley may be greater than a diameter of the end tool pitch pulley.

In an embodiment of the present disclosure, a ratio of the diameter of the end tool pitch pulley to the diameter of the manipulation part pitch wire pulley may range from 1:3.5 to 1:4.5.

In an embodiment of the present disclosure, the end tool pitch pulley may be connected to the manipulation part pitch wire pulley through the pitch wire, and a rotation angle of the end tool pitch pulley when the pitch wire moves may be greater than a rotation angle of the manipulation part pitch wire pulley when the pitch wire moves.

In an embodiment of the present disclosure, a ratio of the rotation angle of the manipulation part pitch wire pulley to the rotation angle of the end tool pitch pulley may range from 1:3.5 to 1:4.5.

According to another aspect of the present invention, there is provided a surgical instrument including an end tool including one or more jaws formed to be rotatable, a manipulation part including a pitch manipulation part configured to control a pitch motion of the end tool, and configured to control a rotation of the end tool, a power transmission part including one or more jaw wires connected to the manipulation part to transmit a rotation of the manipulation part to the one or more jaws, and a connection part formed to extend in a first direction (an X-axis), having one end portion to which the end tool is coupled and another end portion to which the manipulation part is coupled to connect the end tool to the manipulation part, and including a bent part formed to be bent at least once while connecting the end tool to the manipulation part, wherein, in a state in which the end tool is parallel to the first direction, the manipulation part is in a pitch-rotated state at a predetermined rotation angle with respect to the first direction.

In an embodiment of the present disclosure, in a neutral state in which the manipulation part is parallel to the first direction, the end tool may be in a pitch-rotated state at a maximum rotation angle with respect to the first direction.

In an embodiment of the present disclosure, the end tool may include an end tool hub formed to internally accommodate at least some of the jaws, and an end tool pitch pulley formed on an end portion of the end tool hub at a proximal end side, the power transmission part may further include a pitch wire coupled to the end tool pitch pulley to rotate the end tool pitch pulley, the manipulation part may further include a manipulation part pitch wire pulley around which the pitch wire is wound, and a diameter of the manipulation part pitch wire pulley may be greater than a diameter of the end tool pitch pulley.

In an embodiment of the present disclosure, a ratio of the diameter of the end tool pitch pulley to the diameter of the manipulation part pitch wire pulley may range from 1:3.5 to 1:4.5.

In an embodiment of the present disclosure, the end tool pitch pulley may be connected to the manipulation part pitch wire pulley through the pitch wire, and a rotation angle of the end tool pitch pulley when the pitch wire moves may be greater than a rotation angle of the manipulation part pitch wire pulley when the pitch wire moves.

In an embodiment of the present disclosure, a ratio of the rotation angle of the manipulation part pitch wire pulley to the rotation angle of the end tool pitch pulley may range from 1:3.5 to 1:4.5.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A is a conceptual diagram of a pitch motion of a conventional surgical instrument, and FIG. 1B is a conceptual diagram of a yaw motion;

FIG. 1C is a conceptual diagram of a pitch motion of another conventional surgical instrument, and FIG. 1D is a conceptual diagram of a yaw motion;

FIG. 1E is a conceptual diagram of a pitch motion of a surgical instrument according to the present disclosure, and FIG. 1F is a conceptual diagram of a yaw motion;

FIG. 2 is a perspective view illustrating a surgical instrument according to a first embodiment of the present disclosure;

FIG. 3 is a side view of the surgical instrument of FIG. 2;

FIGS. 4 and 5 are perspective views illustrating an end tool of the surgical instrument of FIG. 2;

FIG. 6 is a plan view illustrating the end tool of the surgical instrument of FIG. 2;

FIGS. 7 to 9 are perspective views illustrating the manipulation part of the surgical instrument of FIG. 2;

FIG. 10 is a view schematically illustrating only a configuration of pulleys and wires constituting joints of the surgical instrument illustrated in FIG. 2;

FIG. 11 is a perspective view illustrating a pitch motion of the surgical instrument of FIG. 2;

FIGS. 12 and 13 are diagrams illustrating a configuration of pulleys and wires, which are related to a pitch motion of the surgical instrument illustrated in FIG. 2, in detail for each of the first jaw and the second jaw;

FIG. 14 is a conceptual diagram for describing a pitch motion in relation to a diameter ratio of pitch pulleys of the surgical instrument illustrated in FIG. 2

FIGS. 15 to 17 are side views illustrating a pitch motion of the surgical instrument according to the first embodiment of the present disclosure;

FIGS. 18 and 19 are side views illustrating a pitch motion of a surgical instrument according to a second embodiment of the present disclosure;

FIGS. 20A to 20C are conceptual diagrams illustrating the pitch motion of the surgical instrument of FIG. 17;

FIG. 21 is a side view illustrating a surgical instrument according to a third embodiment of the present disclosure; and

FIG. 22 is a side view illustrating the surgical instrument according to the fourth embodiment of the present disclosure.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Hereinafter, following embodiments will be described in detail with reference to the accompanying drawings, and when the following embodiments are described with reference to the drawings, the same or corresponding components are given the same reference numerals, and repetitive descriptions thereof will be omitted.

As the present embodiments allow for various modifications, particular embodiments will be illustrated in the drawings and further described in the detailed description. The effects and features of the present embodiments and the accompanying methods thereof will become apparent from the following description of the contents, taken in conjunction with the accompanying drawings. However, the present embodiments are not limited to the embodiments disclosed below, but may be implemented in various forms.

In order to clearly describe the present disclosure in the drawings, parts which are not related to the description have been omitted, and like reference numerals refer to similar parts throughout the specification.

In the following embodiments, singular forms are intended to include plural forms as well, unless the context clearly indicates otherwise.

In the following embodiments, terms such as “include” or “have” means that the features or components described in the specification are present, and the possibility that one or more other features or components will be added is not excluded in advance.

In the following embodiments, when a unit, region, or component is referred to as being formed on another unit, region, or component, it can be directly formed on the other unit, region, or component. That is, for example, intervening units, regions, or components may be present.

In the following embodiments, terms such as “connecting” or “coupling” two members do not necessarily mean a direct and/or fixed connection or coupling of the two members, unless the context clearly indicates otherwise, and do not preclude another members from being interposed between the two members.

Sizes of components in the drawings may be exaggerated or reduced for convenience of description. For example, since the size and thickness of each component shown in the drawings are arbitrarily illustrated for convenience of description, the following embodiments are not necessarily limited thereto.

In describing the various embodiments of the present disclosure, it is to be understood that each embodiment is not intended to be interpreted or implemented independently, and that the technical ideas described in each embodiment may be interpreted or implemented in combination with other embodiments described separately.

In a surgical instrument according to the present disclosure, when a manipulation part is rotated in one direction for at least any one of pitch, yaw, and actuation motions, an end tool is rotated in intuitively the same direction as a direction in which the manipulation part is manipulated.

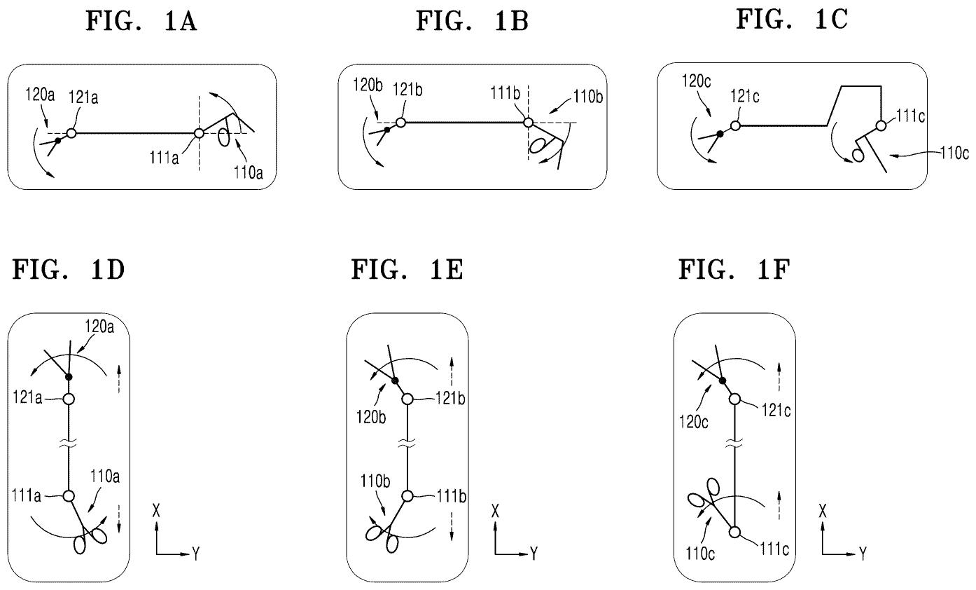

FIG. 1A is a conceptual diagram of a pitch motion of a conventional surgical instrument, and FIG. 1B is a conceptual diagram of a yaw motion.

Referring to FIG. 1A, in performing a pitch motion of a conventional surgical instrument, in a state in which an end tool 120a is formed in front of a rotation center 121a of the end tool, and a manipulation part 110a is formed at the rear of a rotation center 111a of the manipulation part, when the manipulation part 110a is rotated in a clockwise direction, the end tool 120a is also rotated in the clockwise direction, and when the manipulation part 110a is rotated in a counterclockwise direction, the end tool 120a is also rotated in the counterclockwise direction. Referring to FIG. 1B, in performing a yaw motion of the conventional surgical instrument, in a state in which the end tool 120a is formed in front of the rotation center 121a of the end tool, and the manipulation part 110a is formed at the rear of the rotation center 111a of the manipulation part, when the manipulation part 110a is rotated in the clockwise direction, the end tool 120a is also rotated in the clockwise direction, and when the manipulation part 110a is rotated in the counterclockwise direction, the end tool 120a is also rotated in the counterclockwise direction. In this case, in view of left and right directions of a user, when the user moves the manipulation part 110a to the left, the end tool 120a is moved to the right, and when the user moves the manipulation part 110a to the right, the end tool 120a is moved to the left. As a result, a manipulation direction of the user and an operation direction of the end tool are opposite to each other, which may cause the user to make a mistake, and user's manipulation may not be easy.

FIG. 1C is a conceptual diagram of a pitch motion of another conventional surgical instrument, and FIG. 1D is a conceptual diagram of a yaw motion.

Referring to FIG. 1C, in the conventional surgical instrument, which is partially formed in a mirror symmetrical shape, in performing a pitch motion, in a state in which an end tool 120b is formed in front of a rotation center 121b of the end tool, and a manipulation part 110b is formed at the rear of a rotation center 111b of the manipulation part, when the manipulation part 110b is rotated in the clockwise direction, the end tool 120b is rotated in the counterclockwise direction, and when the manipulation part 110b is rotated in the counterclockwise direction, the end tool 120b is rotated in the clockwise direction. In this case, in view of rotation directions of the manipulation part and the end tool, a rotation direction in which the user rotates the manipulation part 110b and a rotation direction of the end tool 120b according thereto are opposite to each other. As a result, the user may be confused with the manipulation direction, and as the operation of the joint is not intuitive, the user may make an error. Further, referring to FIG. 1D, in performing a yaw motion, in a state in which the end tool 120b is formed in front of the rotation center 121b of the end tool, and the manipulation part 110b is formed at the rear of the rotation center 111b of the manipulation part, when the manipulation part 110b is rotated in the clockwise direction, the end tool 120b is rotated in the counterclockwise direction, and when the manipulation part 110b is rotated in the counterclockwise direction, the end tool 120b is rotated in the clockwise direction. In this case, in view of rotation directions of the manipulation part and the end tool, a rotation direction in which the user rotates the manipulation part 110b and a rotation direction of the end tool 120b according thereto are opposite to each other. As a result, the user may be confused with the manipulation direction, and as the operation of the joint is not intuitive, the user may make an error. In the user's pitch or yaw manipulation of the conventional surgical instrument, the user's manipulation direction and the end tool's operation direction do not match each other in view of one of the rotation direction and the left and right directions. This is because the configurations of the end tool and the manipulation part are different from each other in the joint configuration of the conventional surgical instrument. That is, this is because the manipulation part is formed at the rear of the rotation center of the manipulation part, while the end tool is formed in front of the rotation center of the end tool. In order to address the above problems, in a surgical instrument according to an embodiment of the present disclosure, which is illustrated in FIGS. 1E and 1F, an end tool 120c is formed in front of a rotation center 121c of the end tool and a manipulation part 110c is also formed in front of a rotation center 111c of the manipulation part, so that the operations of the manipulation part 110c and the end tool 120c are intuitively matched with each other. In other words, unlike existing examples such as those shown in FIGS. 1A, 1B, 1C, and 1D, in which the manipulation part is close to a user with respect to the joint thereof (that is, away from the end tool), the surgical instrument according to an embodiment of the present disclosure, which is illustrated in FIGS. 1E and 1F, is formed such that at least a portion of the manipulation part is closer (than a joint thereof) to the end tool with respect to the joint thereof at any one moment or more in a manipulation process.

In other words, in the conventional surgical instrument as illustrated in FIGS. 1A, 1B, 1C, and 1D, the manipulation part is formed at the rear of the rotation center thereof, while the end tool is located in front of the rotation center thereof, and thus the end tool is moved at a front side thereof with a rear side fixed through a motion of the manipulation part that is moved at a rear side thereof with a front side thereof fixed, which is not an intuitively matching structure. Accordingly, a mismatch may occur between the manipulation of the manipulation part and the motion of the end tool in view of the left and right directions or in view of the rotation direction, which may cause confusion to the user, and the manipulation of the manipulation part may be difficult to perform intuitively and quickly and may cause mistakes. In contrast, in the surgical instrument according to an embodiment of the present disclosure, since both the end tool and the manipulation part are moved with respect to the rotation center formed at the rear side thereof, it may be said that the motions are intuitively matched with each other in terms of structure. In other words, moving portions of the manipulation part are moved with respect to the rotation center formed at the rear side thereof just as moving portions of the end tool are moved with respect to the rotation center formed at the rear side thereof, and thus it may be said that the motions are intuitively matched with each other in terms of structure. This allows the user to intuitively and quickly perform a control in a direction toward the end tool, and a possibility of making a mistake may be significantly reduced. Hereinafter, a detailed mechanism enabling the above-described function will be described below.

### <First Embodiment of Surgical Instrument>

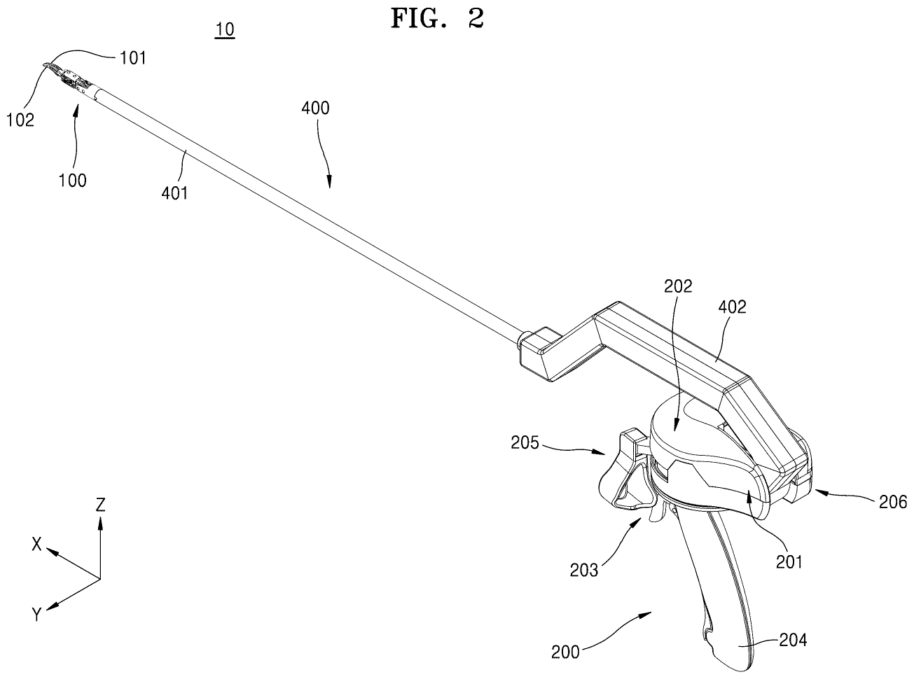

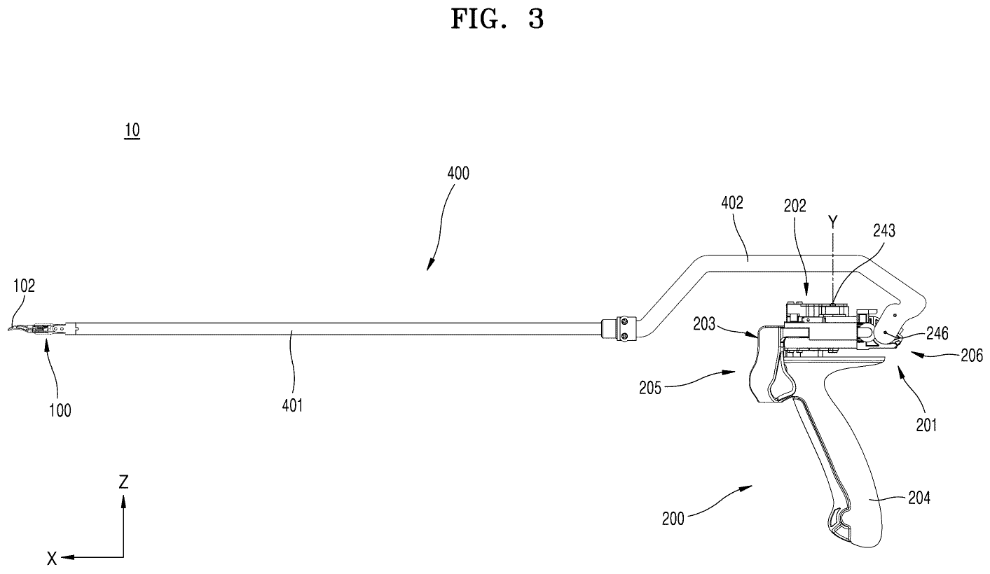

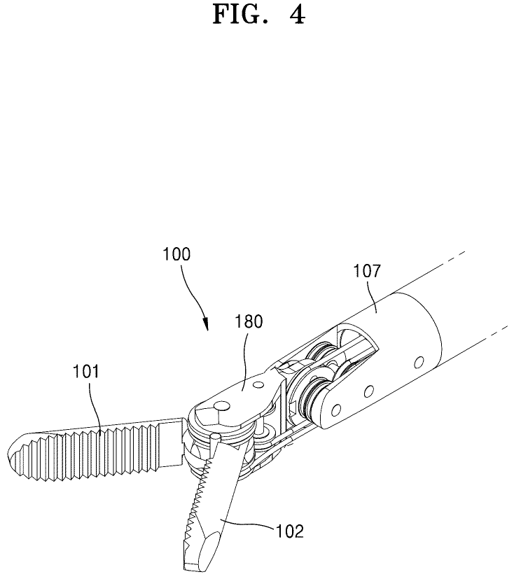

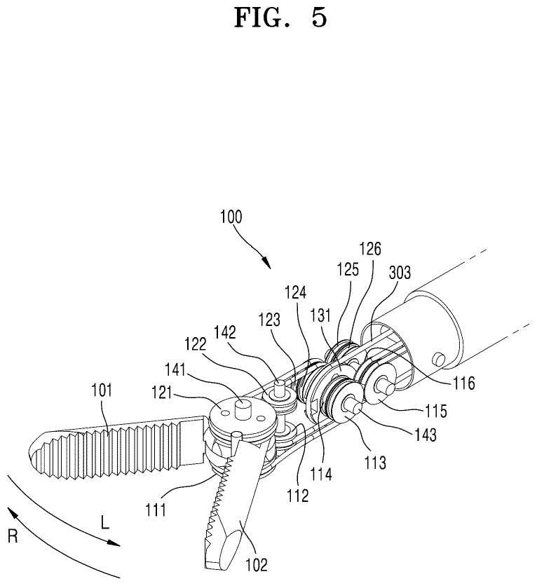

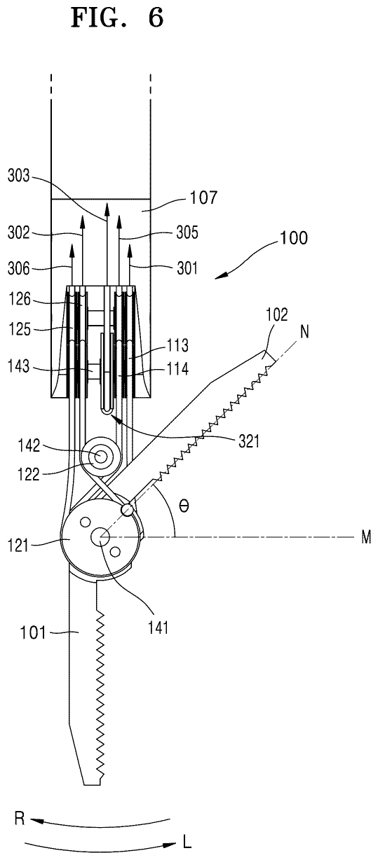

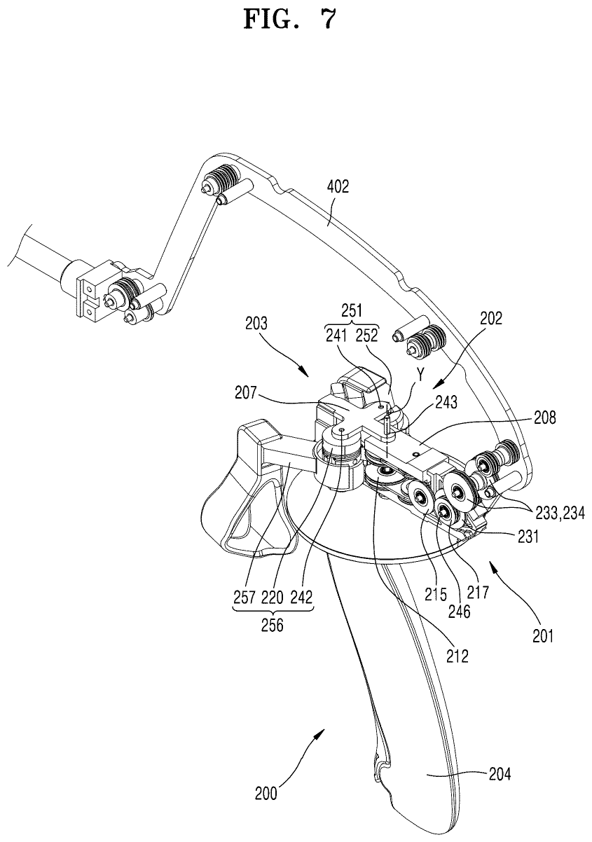

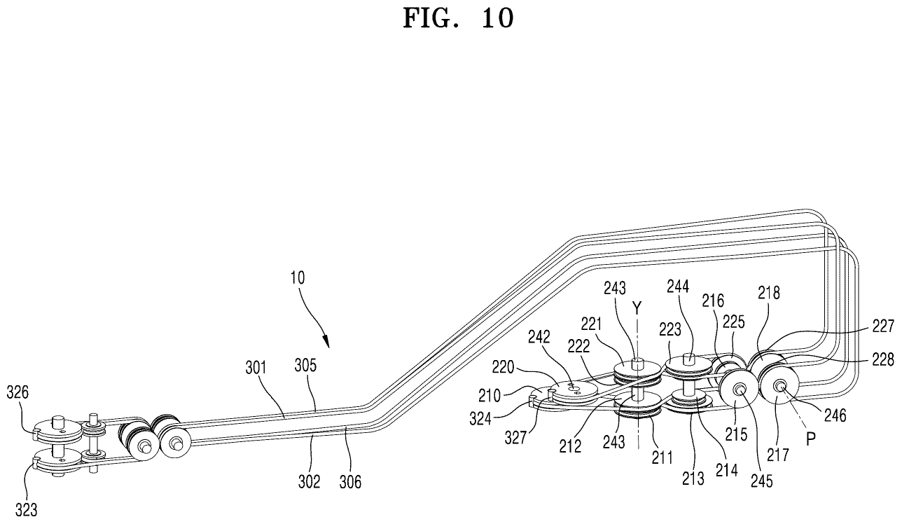

FIG. 2 is a perspective view illustrating a surgical instrument according to a first embodiment of the present disclosure, and FIG. 3 is a side view of the surgical instrument of FIG. 2. FIGS. 4 and 5 are perspective views illustrating the end tool of the surgical instrument illustrated in FIG. 2, and FIG. 6 is a plan view illustrating the end tool of the surgical instrument of FIG. 2. FIGS. 7 to 9 are perspective views illustrating a manipulation part of the surgical instrument illustrated in FIG. 2, and FIG. 10 is a view schematically illustrating a configuration of pulleys and wires constituting joints of the surgical instrument illustrated in FIG. 2.

First, referring to FIGS. 2 and 3, a surgical instrument 10 according to the first embodiment of the present disclosure includes an end tool 100, a manipulation part 200, a power transmission part 300, and a connection part 400.

Here, the connection part 400 is formed in the shape of a hollow shaft, and one or more wires or electric wires may be accommodated therein. The manipulation part 200 is coupled to one end portion of the connection part 400, the end tool 100 is coupled to another end portion thereof, and the connection part 400 may serve to connect the manipulation part 200 to the end tool 100.

Here, the connection part 400 of the surgical instrument 10 according to the first embodiment of the present disclosure includes a straight part 401 and a bent part 402, wherein the straight part 401 is formed at a side coupled to the end tool 100, and the bent part 402 is formed at a side to which the manipulation part 200 is coupled. As such, since the end portion of the connection part 400 at the side of the manipulation part 200 is formed to be bent, a pitch manipulation part 201, a yaw manipulation part 202, and an actuation manipulation part 203 may be formed along an extension line of the end tool 100 or adjacent to the extension line. In other words, it may be said that the pitch manipulation part 201 and the yaw manipulation part 202 are at least partially accommodated in a concave portion formed by the bent part 402. Due to the above-described shape of the bent part 402, the shapes and motions of the manipulation part 200 and the end tool 100 may be further intuitively matched with each other.

Meanwhile, a plane on which the bent part 402 is formed may be substantially the same as a pitch plane, that is, an XZ plane of FIG. 2. As such, as the bent part 402 is formed on the plane substantially the same as the XZ plane, interference with the manipulation part may be reduced. Of course, for intuitive motions of the end tool and the manipulation part, any form other than the XZ plane may be possible.

Meanwhile, a connector (not shown) may be formed on the bent part 402. The connector (not shown) may be connected to an external power source (not shown), and the connector (not shown) may also be connected to the end tool 100 via an electric wire, and may transmit, to the end tool 100, electrical energy supplied from the external power source (not shown).

The manipulation part 200 is formed at the one end portion of the connection part 400 and provided as an interface to be directly controlled by a medical doctor, for example, a tongs shape, a stick shape, a lever shape, or the like, and when the medical doctor controls the manipulation part 200, the end tool 100, which is connected to the interface and inserted into the body of a surgical patient, performs a certain motion, thereby performing surgery. Here, the manipulation part 200 is illustrated in FIG. 2 as being formed in a handle shape that is rotatable while the finger is inserted therein, the concept of the present disclosure is not limited thereto, and various types of manipulation parts that are connected to the end tool 100 and manipulate the end tool 100 may be possible.

The end tool 100 is formed on another end portion of the connection part 400, and performs necessary motions for surgery by being inserted into a surgical site. In an example of the end tool 100, as illustrated in FIG. 2, a pair of jaws 103 for performing a grip motion may be used. However, the concept of the present disclosure is not limited thereto, and various devices for performing surgery may be used as the end tool 100. For example, a configuration of a cantilever cautery may also be used as the end tool. The end tool 100 is connected to the manipulation part 200 by the power transmission part 300, and receives a driving force of the manipulation part 200 through the power transmission part 300 to perform a motion necessary for surgery, such as gripping, cutting, suturing, or the like.

Here, the end tool 100 of the surgical instrument 10 according to the first embodiment of the present disclosure is formed to be rotatable in at least one direction, for example, the end tool 100 may perform a pitch motion around a Y-axis of FIG. 2 and simultaneously perform a yaw motion and an actuation motion around a Z-axis of FIG. 2.

Here, each of the pitch, yaw, and actuation motions used in the present disclosure are defined as follows.

First, the pitch motion means a motion of the end tool 100 rotating in a vertical direction with respect to an extension direction of the connection part 400 (an X-axis direction of FIG. 2), that is, a motion rotating around the Y-axis of FIG. 2. In other words, the pitch motion means a motion of the end tool 100, which is formed to extend from the connection part 400 in the extension direction of the connection part 400 (the X-axis direction of FIG. 2), rotating vertically around the Y-axis with respect to the connection part 400.

Next, the yaw motion means a motion of the end tool 100 rotating in the left and right directions, that is, a motion rotating around the Z-axis of FIG. 2, with respect to the extension direction of the connection part 400 (the X-axis direction of FIG. 2). In other words, the yaw motion means a motion of the end tool 100, which extends from the connection part 400 in the extension direction of the connection part 400 (the X-axis direction of FIG. 2), rotating horizontally around the Z-axis with respect to the connection part 400. That is, the yaw motion means a motion of the two jaws 103, which are formed on the end tool 100, rotating around the Z-axis in the same direction.

Meanwhile, the actuation motion means a motion of the end tool 100 rotating around the same shaft of rotation as that of the yaw motion, while the two jaws 103 rotating in the opposite directions so as to be closed or opened. That is, the actuation motion means a motion of the two jaws 103, which are formed on the end tool 100, rotating in the opposite directions around the Z-axis.

The power transmission part 300 may serve to connect the manipulation part 200 to the end tool 100 to transmit the driving force of the manipulation part 200 to the end tool 100, and may include a plurality of wires, pulleys, links, sections, gears, and the like.

The end tool 100, the manipulation part 200, the power transmission part 300, and the like of the surgical instrument 10 of FIG. 2 will be described in detail later.

### (Intuitive Driving)

Hereinafter, intuitive driving of the surgical instrument 10 of the present disclosure will be described.

First, while holding a first handle 204 with the palm of the hand, the user may rotate the first handle 204 around the Y-axis (i.e., a rotation shaft 246) to perform a pitch motion, and rotate the first handle 204 around the Z-axis (i.e., a rotation shaft 243) to perform a yaw motion. In addition, the user may perform an actuation motion by manipulating the actuation manipulation part 203 while inserting the thumb and the index finger into a first actuation extension part 252 and/or a second actuation extension part 257 in the form of a hand ring formed at one end portion of the actuation manipulation part 203.

Here, in the surgical instrument 10 according to the first embodiment of the present disclosure, when the manipulation part 200 is rotated in one direction with respect to the connection part 400, the end tool 100 is rotated in a direction that is intuitively the same as a manipulation direction of the manipulation part 200. In other words, when the first handle 204 of the manipulation part 200 is rotated in one direction, the end tool 100 is also rotated in a direction intuitively the same as the one direction, so that a pitch motion or a yaw motion is performed. Here, the phrase “intuitively the same direction” may be further explained as meaning that a direction of movement of the user's finger gripping the manipulation part 200 and a direction of movement of a distal end of the end tool 100 form substantially the same direction. Of course, “the same direction” as used herein may not be a perfectly matching direction on a three-dimensional coordinate, and may be understood to be equivalent to the extent that, for example, when the user's finger moves to the left, the distal end of the end tool 100 is moved to the left, and when the user's finger moves down, the end portion of the end tool 100 is moved down.

In addition, to this end, in the surgical instrument 10 according to the first embodiment of the present disclosure, the manipulation part 200 and the end tool 100 are formed in the same direction with respect to a plane perpendicular to the extension axis (X-axis) of the connection part 400. That is, when viewed based on a YZ plane of FIG. 2, the manipulation part 200 is formed to extend in a positive (+) X-axis direction, and the end tool 100 is also formed to extend in the positive (+) X-axis direction. In other words, it may be said that a formation direction of the end tool 100 on one end portion of the connection part 400 is the same as a formation direction of the manipulation part 200 on another end portion of the connection part 400 on the basis of the YZ plane. Further, in other words, it may be said that the manipulation part 200 may be formed in a direction away from the body of a user holding the manipulation part 200, that is, in a direction in which the end tool 100 is formed. That is, in the parts such as the first handle 204, a first actuation manipulation part 251, a second actuation manipulation part 256, and the like, which are moved by the user's grip for actuation, yaw, and pitch motions, a corresponding portion that is moved for the motion is formed to extend in the positive (+) X-axis direction from the rotation center of a corresponding joint for the motion. In this manner, the manipulation part 200 may be configured in the same manner as the end tool 100 in which each moving portion is formed to extend in the positive (+) X-axis direction from the rotation center of a corresponding joint for the motion, and as described with reference to FIG. 1, the manipulation direction of the user may be identical to the operation direction of the end tool from the viewpoint of the rotation directions and the left and right directions. As a result, intuitively the same manipulation may be achieved.

In detail, in the case of the conventional surgical instrument, a direction in which a user manipulates the manipulation part is different from a direction in which the end tool is actually operated, that is, intuitively different from the direction in which the end tool is actually operated, and thus, a surgical operator may not easily intuitively manipulate the surgical instrument and may spend a long time to learn a skill of operating the end tool in desired directions, and in some cases, malfunctions may occur, which may cause damage to patients.

In order to address such problems, the surgical instrument 10 according to the first embodiment of the present disclosure is configured such that the manipulation direction of the manipulation part 200 and the operation direction of the end tool 100 are intuitively identical to each other. To this end, the manipulation part 200 is configured similar to the end tool 100, that is, in the manipulation part 200, portions that are actually moved for actuation, yaw, and pitch motions extend respectively from rotation centers of corresponding joints in the positive (+) X-axis direction.

Hereinafter, the end tool 100, the manipulation part 200, the power transmission part 300, and the like of the surgical instrument 10 of FIG. 2 will be described in more detail.

### (Power Transmission Part)

Hereinafter, the power transmission part 300 of the surgical instrument 10 of FIG. 2 will be described in more detail.

Referring to FIGS. 2 to 14, the power transmission part 300 of the surgical instrument 10 according to an embodiment of the present disclosure may include a wire 301, a wire 302, a wire 303, a wire 304, a wire 305, a wire 306, a wire 307, and a wire 308.

Here, the wire 301 and the wire 305 may be paired to serve as first jaw wires. The wire 302 and the wire 306 may be paired to serve as second jaw wires. Here, the components encompassing the wires 301 and 305, which are first jaw wires, and the wires 302 and 306, which are second jaw wires, may be referred to as jaw wires. In addition, the wire 303 and the wire 304 may be paired to serve as pitch wires.

In addition, the power transmission part 300 of the surgical instrument 10 according to an embodiment of the present disclosure may include a coupling member 321, a coupling member 323, a coupling member 324, a coupling member 326, and a coupling member 327 that are coupled to respective end portions of the wires to respectively couple the wires and the pulleys. Here, each of the coupling members may have various shapes as necessary, such as a ball shape, a tube shape, and the like.

Here, on the end tool 100 side, the coupling member 321 may serve as a pitch wire-end tool coupling member, the coupling member 323 may serve as a first jaw wire-end tool coupling member, and the coupling member 326 may serve as a second jaw wire-end tool coupling member.

In addition, the coupling member 324 serving as a first jaw wire-manipulation part coupling member, and the coupling member 327 serving as a second jaw wire-manipulation part coupling member are formed on the manipulation part 200 side, and although not shown in the drawings, and a pitch wire-manipulation part coupling member may be further formed on the manipulation part 200 side.

The coupling relationship between the wires, the coupling members, and the respectively pulleys will be described in detail as follows.

First, the wires 301 and 305, which are first jaw wires, may be a single wire. The coupling member 323, which is a first jaw wire-end tool coupling member, is inserted at an intermediate point of the first jaw wire, which is a single wire, and the coupling member 323 is crimped and fixed, and then, both strands of the first jaw wire centered on the coupling member 323 may be referred to as the wire 301 and the wire 305, respectively.

Alternatively, the wires 301 and 305, which are first jaw wires, may also be formed as separate wires and connected by the coupling member 323.

In addition, by coupling the coupling member 323 to a pulley 111, the wires 301 and 305 may be fixedly coupled to the pulley 111. This allows the pulley 111 to rotate as the wires 301 and 305 are pulled and released.

Meanwhile, the first jaw wire-manipulation part coupling member 324 may be coupled to another end portions of the wires 301 and 305, which are opposite to one end portions to which the coupling member 323 is coupled.

In addition, by coupling the first jaw wire-manipulation part coupling member 324 to a pulley 210, the wires 301 and 305 may be fixedly coupled to the pulley 210. As a result, when the pulley 210 is rotated by a motor or a human force, the pulley 111 of the end tool 100 may be rotated as the wire 301 and the wire 305 are pulled and released.

In the same manner, the wires 302 and 306, which are second jaw wires, are respectively coupled to the coupling member 326, which is a second jaw wire-end tool coupling member, and the second jaw wire-manipulation part coupling member 327. In addition, the coupling member 326 is coupled to a pulley 121, and the second jaw wire-manipulation part coupling member is coupled to a pulley 220. As a result, when the pulley 220 is rotated by a motor or a human force, the pulley 121 of the end tool 100 may be rotated as the wire 302 and the wire 306 are pulled and released.

In the same manner, the wires 303 and 304, which are pitch wires, are respectively coupled to the coupling member 321, which is a pitch wire-end tool coupling member, and the pitch wire-manipulation part coupling member (not shown). In addition, the coupling member 321 is coupled to a pulley 131, and the pitch wire-manipulation part coupling member (not shown) is coupled to a pulley 231. As a result, when the pulley 231 is rotated by a motor or a human force, the pulley 131 of the end tool 100 may be rotated as the wire 303 and the wire 304 are pulled and released.

### (End Tool)

Hereinafter, the end tool 100 of the surgical instrument 10 of FIG. 2 will be described in more detail.

FIGS. 4 and 5 are perspective views illustrating the end tool of the surgical instrument illustrated in FIG. 2, and FIG. 6 is a plan view illustrating the end tool of the surgical instrument of FIG. 2.

Here, FIG. 4 illustrates a state in which an end tool hub 180 and a pitch hub 107 are coupled to the end tool, and FIG. 5 illustrates a state in which the end tool hub 180 is removed from the end tool. Meanwhile, FIG. 6 mainly illustrates the wires.

Referring to FIGS. 4 to 6 and the like, the end tool 100 of the first embodiment of the present disclosure includes a pair of jaws for performing a grip motion, that is, a first jaw 101 and a second jaw 102. Here, each of the first jaw 101 and the second jaw 102, or a component encompassing the first jaw 101 and the second jaw 102 may be referred to as the jaw 103.

Further, the end tool 100 may include the pulley 111, a pulley 112, a pulley 113, a pulley 114, a pulley 115, and a pulley 116 that are related to a rotational motion of the first jaw 101. In addition, the end tool 100 may include the pulley 121, a pulley 122, a pulley 123, a pulley 124, a pulley 125, and a pulley 126 that are related to a rotational motion of the second jaw 102.

Here, the pulleys facing each other are illustrated in the drawings as being formed parallel to each other, but the concept of the present disclosure is not limited thereto, and each of the pulleys may be variously formed with a position and a size suitable for the configuration of the end tool.

Further, the end tool 100 of the first embodiment of the present disclosure may include the end tool hub 180 and the pitch hub 107.

A rotation shaft 141 and a rotation shaft 142, which will be described later, may be inserted through the end tool hub 180, and the end tool hub 180 may internally accommodate at least some of the pulley 111 and the pulley 121, which are axially coupled to the rotation shaft 141. In addition, the end tool hub 180 may internally accommodate at least some of the pulley 112 and the pulley 122 that are axially coupled to the rotation shaft 142.

Meanwhile, the pulley 131 serving as an end tool pitch pulley may be formed at one end portion of the end tool hub 180. Here, the pulley 131 may be integrally formed with the end tool hub 180 as one body. That is, a disk-shaped pulley is formed at one end portion of the end tool hub 180, and a groove around which a wire may be wound may be formed on an outer circumferential surface of the pulley. Alternatively, the pulley 131 may be formed as a separate member from the end tool hub 180 to be coupled to the end tool hub 180. The wires 303 and 304 described above are coupled to the pulley 131 serving as an end tool pitch pulley, and a pitch motion is performed as the pulley 131 is rotated around a rotation shaft 143.

The rotation shaft 143 and a rotation shaft 144, which will be described later, are inserted through the pitch hub 107, and the pitch hub 107 may be axially coupled to the end tool hub 180 (and the pulley 131) by the rotation shaft 143. Thus, the end tool hub 180 and the pulley 131 may be formed to be rotatable around the rotation shaft 143 with respect to the pitch hub 107.

Further, the pitch hub 107 may internally accommodate at least some of the pulley 113, the pulley 114, the pulley 123, and the pulley 124 that are axially coupled to the rotation shaft 143. In addition, the pitch hub 107 may internally accommodate at least some of the pulley 115, the pulley 116, the pulley 125, and the pulley 126 that are axially coupled to the rotation shaft 144.

Further, the end tool 100 of the first embodiment of the present disclosure may include the rotation shaft 141, the rotation shaft 142, the rotation shaft 143, and the rotation shaft 144. As described above, the rotation shaft 141 and the rotation shaft 142 may be inserted through the end tool hub 180, and the rotation shaft 143 and the rotation shaft 144 may be inserted through the pitch hub 107.

The rotation shaft 141, the rotation shaft 142, the rotation shaft 143, and the rotation shaft 144 may be arranged sequentially from a distal end 104 of the end tool 100 toward a proximal end 105. Accordingly, starting from the distal end 104, the rotation shaft 141 may be referred to as a first pin, the rotation shaft 142 may be referred to as a second pin, the rotation shaft 143 may be referred to as a third pin, and the rotation shaft 144 may be referred to as a fourth pin.

Here, the rotation shaft 141 may function as an end tool jaw pulley rotation shaft, the rotation shaft 142 may function as an end tool jaw auxiliary pulley rotation shaft, the rotation shaft 143 may function as an end tool pitch rotation shaft, and the rotation shaft 144 may function as an end tool pitch auxiliary rotation shaft of the end tool 100.

Each of the rotation shafts 141, 142, 143, and 144 may be fitted into one or more pulleys, which will be described in detail below.

The pulley 111 functions as an end tool first jaw pulley, and the pulley 121 functions as an end tool second jaw pulley. The pulley 111 may also be referred to as a first jaw pulley, and the pulley 121 may also be referred to as a second jaw pulley, and these two components may also be referred to collectively as an end tool jaw pulley.

The pulley 111 and the pulley 121, which are end tool jaw pulleys, are formed to face each other, and are formed to be rotatable independently of each other around the rotation shaft 141, which is an end tool jaw pulley rotation shaft. Here, in the drawings, it is illustrated that the pulley 111 and the pulley 121 are formed to rotate around one rotation shaft 141, but it is of course possible that each end tool jaw pulley may be formed to be rotatable around a separate shaft. Here, the first jaw 101 is fixedly coupled to the pulley 111 and rotates together with the pulley 111, and the second jaw 102 is fixedly coupled to the pulley 121 and rotates together with the pulley 121. Yaw and actuation motions of the end tool 100 are performed in response to the rotation of the pulley 111 and the pulley 121. That is, when the pulley 111 and the pulley 121 are rotated in the same direction around the rotation shaft 141, the yaw motion is performed, and when the pulley 111 and the pulley 121 are rotated in opposite directions around the rotation shaft 141, the actuation motion is performed.

Here, the first jaw 101 and the pulley 111 may be formed as separate members and coupled to each other, or the first jaw 101 and the pulley 111 may be integrally formed as one body. Similarly, the second jaw 102 and the pulley 121 may be formed as separate members and coupled to each other, or the second jaw 102 and the pulley 121 may be integrally formed as one body.

The pulley 112 functions as an end tool first jaw auxiliary pulley, and the pulley 122 functions as an end tool second jaw auxiliary pulley, and these two components may be collectively referred to as an end tool jaw auxiliary pulley or simply an auxiliary pulley.

In detail, the pulley 112 and the pulley 122, which are end tool jaw auxiliary pulleys, may be additionally provided on one side of the pulley 111 and one side the pulley 121, respectively. In other words, the pulley 112, which is an auxiliary pulley, may be disposed between the pulley 111 and the pulley 113/pulley 114. In addition, the pulley 122, which is an auxiliary pulley, may be disposed between the pulley 121 and the pulley 123 or the pulley 124. The pulley 112 and the pulley 122 may be formed to be rotatable independently of each other around the rotation shaft 142. Here, in the drawings, it is illustrated that the pulley 112 and the pulley 122 are formed to rotate around one rotation shaft 142, but it is of course possible that each of the pulley 112 and the pulley 122 may be formed to be rotatable around a separate shaft.

The pulley 113 and the pulley 114 function as end tool first jaw pitch main pulleys, and the pulley 123 and the pulley 124 function as end tool second jaw pitch main pulleys, and these two components may be referred to collectively as an end tool jaw pitch main pulley.

The pulley 115 and the pulley 116 function as end tool first jaw pitch sub-pulleys, and the pulley 125 and the pulley 126 function as end tool second jaw pitch sub-pulleys, and these two components may be collectively referred to as an end tool jaw pitch sub-pulley.

Hereinafter, components related to the rotation of the pulley 111 will be described.

The pulley 113 and the pulley 114 function as end tool first jaw pitch main pulleys. That is, the pulley 113 and the pulley 114 function as main rotation pulleys for a pitch motion of the first jaw 101. Here, the wire 301, which is a first jaw wire, is wound around the pulley 113, and the wire 305, which is a first jaw wire, is wound around the pulley 114.

The pulley 115 and the pulley 116 function as end tool first jaw pitch sub-pulleys. That is, the pulley 115 and the pulley 116 function as sub rotation pulleys for a pitch motion of the first jaw 101. Here, the wire 301, which is a first jaw wire, is wound around the pulley 115, and the wire 305, which is a first jaw wire, is wound around the pulley 116.

Here, the pulley 113 and the pulley 114 are disposed on one side of the pulley 111 and the pulley 112 to face each other. Here, the pulley 113 and the pulley 114 are formed to be rotatable independently of each other around the rotation shaft 143 that is an end tool pitch rotation shaft. In addition, the pulley 115 and the pulley 116 are disposed on one side of the pulley 113 and one side of the pulley 114, respectively, to face each other. Here, the pulley 115 and the pulley 116 are formed to be rotatable independently of each other around the rotation shaft 144 that is an end tool pitch auxiliary rotation shaft. Here, in the drawings, it is illustrated that the pulley 113, the pulley 115, the pulley 114, and the pulley 116 are all formed to be rotatable around the Y-axis direction, but the concept of the present disclosure is not limited thereto, and the rotation shafts of the respective pulleys may be formed in various directions according to configurations thereof.

The wire 301, which is a first jaw wire, is sequentially wound to make contact with at least portions of the pulley 115, the pulley 113, and the pulley 111. In addition, the wire 305 connected to the wire 301 by the coupling member 323 is sequentially wound to make contact with at least portions of the pulley 111, the pulley 112, the pulley 114, and the pulley 116.

In other words, the wires 301 and 305, which are first jaw wires, are sequentially wound to make contact with at least portions of the pulley 115, the pulley 113, the pulley 111, the pulley 112, the pulley 114, and the pulley 116 and are formed to move along the above pulleys while rotating the above pulleys.

Accordingly, when the wire 301 is pulled in the direction of an arrow 301 of FIG. 6, the coupling member 323 to which the wire 301 is coupled and the pulley 111 coupled to the coupling member 323 are rotated in an arrow L direction of FIG. 6. In contrast, when the wire 305 is pulled in the direction of an arrow 305 of FIG. 6, the coupling member 323 to which the wire 305 is coupled and the pulley 111 coupled to the coupling member 323 are rotated in an arrow R direction of FIG. 6.

Next, components related to the rotation of the pulley 121 will be described.

The pulley 123 and the pulley 124 function as end tool second jaw pitch main pulleys. That is, the second jaw 102 functions as a main rotation pulley for a pitch motion of the second jaw 102. Here, the wire 306, which is a second jaw wire, is wound around the pulley 123, and the wire 302, which is a second jaw wire, is wound around the pulley 124.

The pulley 125 and the pulley 126 function as end tool second jaw sub-pulleys. That is, the pulley 125 and the pulley 126 function as sub rotation pulleys for a pitch motion of the second jaw 102. Here, the wire 306, which is a second jaw wire, is wound around the pulley 125, and the wire 302, which is a second jaw wire, is wound around the pulley 126.

On one side of the pulley 121, the pulley 123 and the pulley 124 are disposed to face each other. Here, the pulley 123 and the pulley 124 are formed to be rotatable independently of each other around the rotation shaft 143 which is an end tool pitch rotation shaft. In addition, the pulley 125 and the pulley 126 are disposed on one side of the pulley 123 and one side of the pulley 124, respectively, to face each other. Here, the pulley 125 and the pulley 126 are formed to be rotatable independently of each other around the rotation shaft 144 that is an end tool pitch auxiliary rotation shaft. Here, in the drawings, it is illustrated that all of the pulley 123, the pulley 125, the pulley 124, and the pulley 126 are formed to be rotatable around the Y-axis direction, but the concept of the present disclosure is not limited thereto, and the rotation axes of the respective pulleys may be formed in various directions according to configurations thereof.

The wire 306, which is a second jaw wire, is sequentially wound to make contact with at least portions of the pulley 125, the pulley 123, and the pulley 121. In addition, the wire 302 connected to the wire 306 by the coupling member 326 is sequentially wound to make contact with at least portions of the pulley 121, the pulley 122, the pulley 124, and the pulley 126.

In other words, the wires 306 and 302, which are second jaw wires, are sequentially wound to make contact with at least portions of the pulley 125, the pulley 123, the pulley 121, the pulley 122, the pulley 124, and the pulley 126, and are formed to move along the above pulleys while rotating the above pulleys.

Accordingly, when the wire 306 is pulled in the direction of an arrow 306 of FIG. 6, a coupling member 326 to which the wire 306 is coupled and the pulley 121 coupled to the coupling member 326 are rotated in the arrow R direction of FIG. 6. In contrast, when the wire 302 is pulled in the direction of an arrow 302 of FIG. 6, the coupling member 326 to which the wire 302 is coupled and the pulley 121 coupled to the coupling member 326 are rotated in the arrow L direction of FIG. 6.

Hereinafter, a pitch motion of the present disclosure will be described in more detail.

Meanwhile, when the wire 301 is pulled toward the arrow 301 of FIG. 6, and simultaneously, the wire 305 is pulled toward the arrow 305 of FIG. 6 (that is, when both strands of the first jaw wire are pulled), as shown in FIG. 5, since the wires 301 and 305 are wound around lower portions of the pulley 113 and the pulley 114 rotatable around the rotation shaft 143, which is an end tool pitch rotation shaft, the pulley 111 to which the wires 301 and 305 are fixedly coupled and the end tool hub 180 to which the pulley 111 is coupled are rotated as a whole in the counterclockwise direction around the rotation shaft 143, so that the end tool 100 is rotated downward to perform a pitch motion. At this time, since the second jaw 102 and the wires 302 and 306 fixedly coupled thereto are wound around upper portions of the pulley 123 and the pulley 124 rotatable around the rotation shaft 143, the wires 302 and 306 are unwound in opposite directions of the arrows 302 and 306, respectively.

In contrast, when the wire 302 is pulled toward the arrow 302 of FIG. 6, and simultaneously, the wire 306 is pulled toward the arrow 306 of FIG. 6, as shown in FIG. 5, since the wires 302 and 306 are wound around the upper portions of the pulley 123 and the pulley 124 rotatable around the rotation shaft 143, which is an end tool pitch rotation shaft, the pulley 121 to which the wires 302 and 306 are fixedly coupled and the end tool hub 180 to which the pulley 121 is coupled are rotated as a whole in the clockwise direction around the rotation shaft 143, so that the end tool 100 is rotated upward to perform a pitch motion. At this time, since the first jaw 101 and the wires 301 and 305 fixedly coupled thereto are wound around lower portions of the pulley 113 and the pulley 114 rotatable around the rotation shaft 143, the wires 302 and 306 are moved in opposite directions of the wires 301 and 305, respectively.

Meanwhile, the end tool 100 of the surgical instrument 10 of the present disclosure may further include the pulley 131, which is an end tool pitch pulley, the manipulation part 200 may further include the pulley 231 and a pulley 232, which are manipulation part pitch wire pulleys, and the power transmission part 300 may further include the wires 303 and 304, which are pitch wires. In detail, the pulley 131 of the end tool 100 is rotatable around the rotation shaft 143, which is an end tool pitch rotation shaft, and may be integrally formed with the end tool hub 180 (or fixedly coupled to the end tool hub 180) as one body. In addition, the wires 303 and 304 may serve to connect the pulley 131 of the end tool 100 to the pulley 231 and the pulley 232 of the manipulation part 200.

Thus, when the pulley 231 and the pulley 232 of the manipulation part 200 are rotated, the rotation of the pulley 231 and the pulley 232 is transmitted to the pulley 131 of the end tool 100 through the wires 303 and 304 so that the pulley 131 is rotated together therewith, and as a result, the end tool 100 performs a pitch motion while rotating.

That is, in the surgical instrument 10 according to the first embodiment of the present disclosure, by providing the pulley 131 of the end tool 100, the pulley 231 and the pulley 232 of the manipulation part 200, and the wires 303 and 304 of the power transmission part 300 to transmit power for a pitch motion, the driving force for the pitch motion of the manipulation part 200 may be more completely transmitted to the end tool 100, thereby improving operation reliability.

Here, a diameter of each of the pulley 113, the pulley 114, the pulley 123, and the pulley 124, which are end tool jaw pitch main pulleys, and a diameter of the pulley 131, which is an end tool pitch pulley, may be the same as each other or different from each other. At this time, a ratio of the diameter of the end tool jaw pitch main pulley to the diameter of the end tool pitch pulley may be the same as a ratio of a diameter of the manipulation part pitch main pulley of the manipulation part 200 to a diameter of a manipulation part pitch wire pulley to be described later. This will be described in detail below.

### (Manipulation Part)

FIGS. 7 to 9 are perspective views illustrating the manipulation part of the surgical instrument of FIG. 2. FIG. 10 is a view schematically illustrating only a configuration of pulleys and wires constituting joints of the surgical instrument illustrated in FIG. 2.

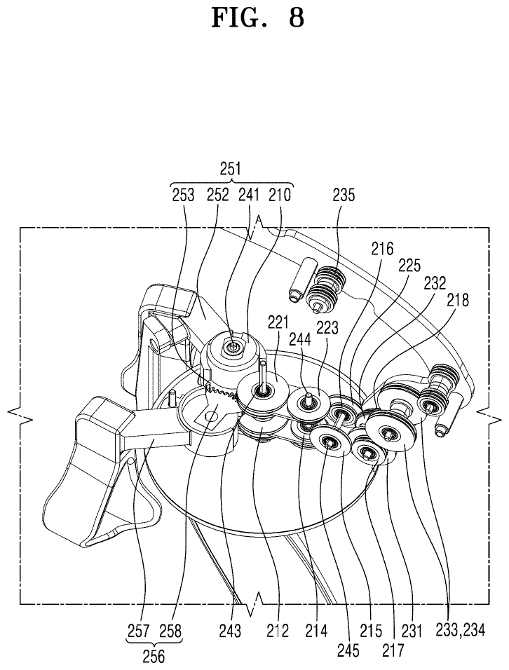

Referring to FIGS. 2 to 10, the manipulation part 200 of the surgical instrument 10 according to the first embodiment of the present disclosure includes the first handle 204 that a user can grip, the actuation manipulation part 203 configured to control an actuation motion of the end tool 100, the yaw manipulation part 202 configured to control a yaw motion of the end tool 100, and the pitch manipulation part 201 configured to control a pitch motion of the end tool 100. Here, it is understood that only the components related to the pitch/yaw/actuation motions of the surgical instrument 10 are illustrated in FIGS. 7 and 8.

The manipulation part 200 may include the pulley 210, a pulley 211, a pulley 212, a pulley 213, a pulley 214, a pulley 215, a pulley 216, a pulley 217, and a pulley 218 that are related to a rotational motion of the first jaw 101. In addition, the manipulation part 200 may include the pulley 220, a pulley 221, a pulley 222, a pulley 223, a pulley 224, a pulley 225, a pulley 226, a pulley 227, and a pulley 228 that are related to a rotational motion of the second jaw 102. In addition, the manipulation part 200 may include the pulley 231, the pulley 232, a pulley 233, and a pulley 234 that are related to a pitch motion of the end tool 100. In addition, the manipulation part 200 may include a pulley 235, which is a relay pulley disposed at some places along the bent part 402 of the connection part 400.

Here, the pulleys facing each other are illustrated in the drawings as being formed parallel to each other, but the concept of the present disclosure is not limited thereto, and each of the pulleys may be variously formed with a position and a size suitable for the configuration of the manipulation part.

Further, the manipulation part 200 of the first embodiment of the present disclosure may include a rotation shaft 241, a rotation shaft 242, the rotation shaft 243, a rotation shaft 244, a rotation shaft 245, and the rotation shaft 246. Here, the rotation shaft 241 may function as a manipulation part first jaw actuation rotation shaft, and the rotation shaft 242 may function as a manipulation part second jaw actuation rotation shaft. In addition, the rotation shaft 243 may function as a manipulation part yaw main rotation shaft, and the rotation shaft 244 may function as a manipulation part yaw sub-rotation shaft. In addition, the rotation shaft 245 may function as a manipulation part pitch sub-rotation shaft, and the rotation shaft 246 may function as a manipulation part pitch main rotation shaft.

The rotation shaft 241/rotation shaft 242, the rotation shaft 243, the rotation shaft 244, the rotation shaft 245, and the rotation shaft 246 may be sequentially disposed from a distal end 205 of the manipulation part 200 toward a proximal end 206.

Each of the rotation shafts 241, 242, 243, 244, 245, and 246 may be fitted into one or more pulleys, which will be described in detail later.

The pulley 210 functions as a manipulation part first jaw actuation pulley, the pulley 220 functions as a manipulation part second jaw actuation pulley, and these components may also be collectively referred to as a manipulation part actuation pulley.

The pulley 211 and the pulley 212 function as manipulation part first jaw yaw main pulleys, the pulley 221 and the pulley 222 function as manipulation part second jaw yaw main pulleys, and these components may also be collectively referred to as a manipulation part yaw main pulley.

The pulley 213 and the pulley 214 function as manipulation part first jaw yaw sub-pulleys, the pulley 223 and the pulley 224 function as manipulation part second jaw yaw sub-pulleys, and these components may also be collectively referred to as a manipulation part yaw sub-pulley.

The pulley 215 and the pulley 216 function as manipulation part first jaw pitch sub-pulleys, the pulley 225 and the pulley 226 function as manipulation part second jaw pitch sub-pulleys, and these components may also be collectively referred to as a manipulation part pitch sub-pulley.

The pulley 217 and the pulley 218 function as manipulation part first jaw pitch main pulleys, and the pulley 227 and the pulley 228 function as manipulation part second jaw pitch main pulleys, and these components may also be collectively referred to as a manipulation part pitch main pulley.

The pulley 231 and the pulley 232 function as manipulation part pitch wire main pulleys, and the pulley 233 and the pulley 234 function as manipulation part pitch wire auxiliary pulleys.

The above components are categorized from the perspective of the manipulation part for each motion (pitch/yaw/actuation) as follows.

The pitch manipulation part 201 configured to control a pitch motion of the end tool 100 may include the pulley 215, the pulley 216, the pulley 217, the pulley 218, the pulley 225, the pulley 226, the pulley 227, the pulley 228, the pulley 231, the pulley 232, and the pulley 234. In addition, the pitch manipulation part 201 may include the rotation shaft 245 and the rotation shaft 246. In addition, the pitch manipulation part 201 may further include a pitch frame 208.

The yaw manipulation part 202 configured to control a yaw motion of the end tool 100 may include the pulley 211, the pulley 212, the pulley 213, the pulley 214, the pulley 221, the pulley 222, the pulley 223, and the pulley 224. In addition, the yaw manipulation part 202 may include the rotation shaft 243 and the rotation shaft 244. In addition, the yaw manipulation part 202 may further include a yaw frame 207.

The actuation manipulation part 203 configured to control an actuation motion of the end tool 100 may include the pulley 210, the pulley 220, the rotation shaft 241, and the rotation shaft 242. In addition, the actuation manipulation part 203 may further include the first actuation manipulation part 251 and the second actuation manipulation part 256.

Hereinafter, each component of the manipulation part 200 will be described in more detail.

The first handle 204 may be formed to be gripped by a user with the hand, and in particular, may be formed to be grasped by the user by wrapping the first handle 204 with his/her palm. In addition, the actuation manipulation part 203 and the yaw manipulation part 202 are formed on the first handle 204, and the pitch manipulation part 201 is formed on one side of the yaw manipulation part 202. In addition, another end portion of the pitch manipulation part 201 is connected to the bent part 402 of the connection part 400.

The actuation manipulation part 203 includes the first actuation manipulation part 251 and the second actuation manipulation part 256. The first actuation manipulation part 251 includes the rotation shaft 241, the pulley 210, the first actuation extension part 252, and a first actuation gear 253. The second actuation manipulation part 256 includes the rotation shaft 242, the pulley 220, the second actuation extension part 257, and a second actuation gear 258. Here, end portions of the first actuation extension part 252 and the second actuation extension part 257 are each formed in the shape of a hand ring, and may act as a second handle.

Here, the rotation shaft 241 and the rotation shaft 242, which are actuation rotation axes, may be formed to form a predetermined angle with an XY plane on which the connection part 400 is formed. For example, the rotation shaft 241 and the rotation shaft 242 may be formed in a direction parallel to the Z-axis, and in this state, when the pitch manipulation part 201 or the yaw manipulation part 202 is rotated, a coordinate system of the actuation manipulation part 203 may change relatively. Of course, the concept of the present disclosure is not limited thereto, and the rotation shaft 241 and the rotation shaft 242 may be formed in various directions so as to be suitable for a structure of the hand of the user gripping the actuation manipulation part 203 according to an ergonomic design.

Meanwhile, the pulley 210, the first actuation extension part 252, and the first actuation gear 253 are fixedly coupled to each other to be rotatable together around the rotation shaft 241. Here, the pulley 210 may be configured as a single pulley or two pulleys fixedly coupled to each other.

Similarly, the pulley 220, the second actuation extension part 257, and the second actuation gear 258 are fixedly coupled to each other to be rotatable together around the rotation shaft 242. Here, the pulley 220 may be configured as a single pulley or two pulleys fixedly coupled to each other.

Here, the first actuation gear 253 and the second actuation gear 258 are formed to be engaged with each other such that, when any one gear is rotated in one direction, another gear is rotated together with the one gear in a direction opposite to the one direction.

The yaw manipulation part 202 may include the rotation shaft 243, the pulleys 211 and 212, which are manipulation part first jaw yaw main pulleys, the pulleys 221 and 222, which are manipulation part second jaw yaw main pulleys, and the yaw frame 207. In addition, the yaw manipulation part 202 may further include the pulleys 213 and 214, which are manipulation part first jaw yaw sub-pulleys formed on one side of the pulley 211 and one side of the pulley 212, respectively, and the pulleys 223 and 224 that are manipulation part second jaw yaw sub-pulleys formed on one side of the pulley 221 and one side of the pulley 222, respectively. Here, the pulleys 213 and 214 and the pulleys 223 and 224 may be coupled to the pitch frame 208 to be described later.

Here, it is illustrated in the drawings that the yaw manipulation part 202 includes the pulleys 211 and 212 and the pulleys 221 and 222, wherein the pulleys 211 and 212 and the pulleys 221 and 222 are each provided with two pulleys formed to face each other and independently rotatable, but the concept of the present disclosure is not limited thereto. That is, one or more pulleys having the same diameter or different diameters may be provided according to the configuration of the yaw manipulation part 202.

In detail, the rotation shaft 243, which is a manipulation part yaw main rotation shaft, is formed on one side of the actuation manipulation part 203 on the first handle 204. At this time, the first handle 204 is formed to be rotatable around the rotation shaft 243.

Here, the rotation shaft 243 may be formed to form a predetermined angle with the XY plane on which the connection part 400 is formed. For example, the rotation shaft 243 may be formed in a direction parallel to the Z-axis, and in this state, when the pitch manipulation part 201 is rotated, a coordinate system of the rotation shaft 243 may change relatively as described above. Of course, the concept of the present disclosure is not limited thereto, and the rotation shaft 243 may be formed in various directions so as to be suitable for a structure of the hand of the user gripping the manipulation part 200 according to an ergonomic design.

Meanwhile, the pulleys 211 and 212 and the pulleys 221 and 222 are coupled to the rotation shaft 243 so as to be rotatable around the rotation shaft 243. In addition, the wire 301 or the wire 305, which is a first jaw wire, is wound around the pulleys 211 and 212, and the wire 302 or the wire 306, which is a second jaw wire, may be wound around the pulleys 221 and 222. In this case, the pulleys 211 and 212 and the pulleys 221 and 222 may each be configured as two pulleys formed to face each other and independently rotatable. Accordingly, a wire being wound and a wire being released may be wound around respective separate pulleys so that the wires may perform motions without interference with each other.

The yaw frame 207 rigidly connects the first handle 204, the rotation shaft 241, the rotation shaft 242, and the rotation shaft 243 to each other, so that the first handle 204, the yaw manipulation part 202, and the actuation manipulation part 203 are integrally yaw-rotated around the rotation shaft 243.

The pitch manipulation part 201 may include the rotation shaft 246, the pulley 217 and the pulley 218, which are manipulation part first jaw pitch main pulleys, the pulleys 227 and 228, which are manipulation part second jaw pitch main pulleys, and the pitch frame 208. In addition, the pitch manipulation part 201 may further include the rotation shaft 245, the pulleys 215 and 216, which are manipulation part first jaw pitch sub-pulleys formed on one side of the pulley 217 and one side of the pulley 218, respectively, and the pulleys 225 and 226, which are manipulation part second jaw pitch sub-pulleys formed on one side of the pulley 227 and one side of the pulley 228, respectively. The pitch manipulation part 201 may be connected to the bent part 402 of the connection part 400 through the rotation shaft 246.

In detail, the pitch frame 208 is a base frame of the pitch manipulation part 201, and the rotation shaft 243 is rotatably coupled to one end portion thereof. That is, the yaw frame 207 is formed to be rotatable around the rotation shaft 243 with respect to the pitch frame 208.

As described above, since the yaw frame 207 connects the first handle 204, the rotation shaft 243, the rotation shaft 241, and the rotation shaft 242 to each other, and the yaw frame 207 is also axially coupled to the pitch frame 208, when the pitch frame 208 is pitch-rotated around the rotation shaft 246, the yaw frame 207 connected to the pitch frame 208, the first handle 204, the rotation shaft 241, the rotation shaft 242, and the rotation shaft 243 are pitch-rotated together with the pitch frame 208. That is, when the pitch manipulation part 201 is rotated around the rotation shaft 246, the actuation manipulation part 203 and the yaw manipulation part 202 are rotated together with the pitch manipulation part 201. In other words, when a user pitch-rotates the first handle 204 around the rotation shaft 246, the actuation manipulation part 203, the yaw manipulation part 202, and the pitch manipulation part 201 are moved together with the first handle 204.

The pulleys 217 and 218 and the pulleys 227 and 228 are coupled to the rotation shaft 246 so as to be rotatable around the rotation shaft 246 of the pitch frame 208.

Here, the pulley 217 and the pulley 218 may be formed to face each other so as to be independently rotatable. Accordingly, a wire being wound and a wire being released may be wound around respective separate pulleys so that the wires may perform motions without interference with each other. Similarly, the pulley 227 and the pulley 228 may also be formed to face each other so as to be independently rotatable. Accordingly, a wire being wound and a wire being released may be wound around respective separate pulleys so that the wires may perform motions without interference with each other.

Next, a motion of each of the wires 303 and 304, which are pitch wires, is described as follows.

The pulley 131, which is an end tool pitch pulley, is fixedly coupled to the end tool hub 180 in the end tool 100, and the pulley 231 and the pulley 232, which are manipulation part pitch wire pulleys, are fixedly coupled to the pitch frame 208 in the manipulation part 200. In addition, these pulleys are connected to each other by the wires 303 and 304, which are pitch wires, so that a pitch motion of the end tool 100 may be performed more easily according to the pitch manipulation of the manipulation part 200. Here, the wire 303 is fixedly coupled to the pitch frame 208 via the pulley 231 and the pulley 233, and the wire 304 is fixedly coupled to the pitch frame 208 via the pulley 232 and the pulley 234. That is, the pitch frame 208 and the pulleys 231 and 232 are rotated together around the rotation shaft 246 by the pitch rotation of the manipulation part 200, and as a result, the wires 303 and 304 are also moved, and thus, a driving force of additional pitch rotation may be transmitted separately from the pitch motion of the end tool by the wire 301, the wire 302, the wire 305, and the wire 306, which are jaw wires.

A connection relationship of each of the first handle 204, the pitch manipulation part 201, the yaw manipulation part 202, and the actuation manipulation part 203 is summarized as follows. The rotation shafts 241 and 242, the rotation shaft 243, the rotation shaft 244, the rotation shaft 245, and the rotation shaft 246 may be formed on the first handle 204. In this case, since the rotation shafts 241 and 242 are directly formed on the first handle 204, the first handle 204 and the actuation manipulation part 203 may be directly connected to each other. Meanwhile, since the rotation shaft 243 is directly formed on the first handle 204, the first handle 204 and the yaw manipulation part 202 may be directly connected to each other. On the other hand, since the pitch manipulation part 201 is formed on one side of the yaw manipulation part 202 so as to be connected to the yaw manipulation part 202, the pitch manipulation part 201 is not directly connected to the first handle 204, and the pitch manipulation part 201 and the first handle 204 may be formed to be indirectly connected to each other via the yaw manipulation part 202.

Continuing to refer to the drawings, in the surgical instrument 10 according to the first embodiment of the present disclosure, the pitch manipulation part 201 and the end tool 100 may be formed on the same or parallel axis (X-axis). That is, the rotation shaft 246 of the pitch manipulation part 201 is formed at one end portion of the bent part 402 of the connection part 400, and the end tool 100 is formed at another end portion of the connection part 400.

In addition, one or more relay pulleys 235 configured to change or guide paths of the wires may be disposed at some places along the connection part 400, particularly in the bent part 402. As at least some of the wires are wound around the relay pulleys 235 to guide the paths of the wires, these wires may be disposed along a bent shape of the bent part 402.

Here, in the drawings, it is illustrated that the connection part 400 is formed to be curved with a predetermined curvature by having the bent part 402, but the concept of the present disclosure is not limited thereto, and the connection part 400 may be formed linearly or to be bent one or more times as necessary, and even in this case, it may be said that the pitch manipulation part 201 and the end tool 100 are formed on substantially the same axis or parallel axes. In addition, although FIG. 3 illustrates that each of the pitch manipulation part 201 and the end tool 100 is formed on an axis parallel to the X-axis, the concept of the present disclosure is not limited thereto, and the pitch manipulation part 201 and the end tool 100 may be formed on different axes.

Meanwhile, when a bent part, that is, an arc-shaped frame, connecting the connection part to the manipulation part is formed to be excessively elevated, interference between instrument frames may occur when a user grips the surgical instruments one by one with both hands while using the surgical instruments simultaneously.

In the surgical instrument according to the first embodiment of the present disclosure, the height of the arc-shaped frame is reduced to minimize interference between a plurality of instruments when used simultaneously while not affecting functionality.

To this end, a pitch motion ratio of the end tool to the manipulation part is increased so that the manipulation part does not interfere with the arc-shaped frame when the end tool is fully rotated. That is, the height of the arc-shaped frame may be reduced by reducing the amount of rotation of the manipulation part by allowing the end tool to rotate larger even when the manipulation part rotates less.

To this end, the amount of rotation of the manipulation part may be controlled by varying a diameter of the pulley on the end tool related to the pitch motion and a diameter of the pulley on the manipulation part related to the pitch motion, which will be described in more detail later.

### (Actuation, Yaw, and Pitch Motions)

Actuation, yaw, and pitch motions in the present embodiment will be described as follows.

First, the actuation motion will be described below.

In a state in which a user inserts his/her index finger in the hand ring formed on the first actuation extension part 252 and his/her thumb in the hand ring formed on the second actuation extension part 257, when the user rotates the actuation extension parts 252 and 257 using one or both of his/her index finger and thumb, the pulley 210 and the first actuation gear 253 fixedly coupled to the first actuation extension part 252 are rotated around the rotation shaft 241, and the pulley 220 and the second actuation gear 258 fixedly coupled to the second actuation extension part 257 are rotated around the rotation shaft 242. At this time, the pulley 210 and the pulley 220 are rotated in opposite directions, and thus the wires 301 and 305 fixedly coupled to the pulley 210 at one end portion thereof and the wires 302 and 306 fixedly coupled to the pulley 220 at one end portion thereof are also moved in opposite directions. In addition, a rotating force is transmitted to the end tool 100 through the power transmission part 300, and two jaws 103 of the end tool 100 perform the actuation motion.

Here, as described above, the actuation motion refers to a motion in which the two jaws 101 and 102 are splayed or closed while being rotated in opposite directions. That is, when the actuation extension parts 252 and 257 of the actuation manipulation part 203 are rotated in directions close to each other, the first jaw 101 is rotated in the counterclockwise direction, and the second jaw 102 is rotated in the clockwise direction, thereby closing the end tool 100. That is, when the actuation extension parts 252 and 257 of the actuation manipulation part 203 are rotated in directions away from each other, the first jaw 101 is rotated in the counterclockwise direction, and the second jaw 102 is rotated in the clockwise direction, thereby opening the end tool 100.

In the present embodiment, for the actuation manipulation described above, the first actuation extension part 252 and the second actuation extension part 257 are provided to configure the second handle and manipulated by gripping the second handle with two fingers. However, for the actuation manipulation in which the two jaws of the end tool 100 are opened or closed, the actuation manipulation part 203 may be configured in a manner different from the above-described manner, such as configuring the two actuation pulleys (the pulley 210 and the pulley 220) to act in opposition to each other with one actuation rotation part.

Next, the yaw motion will be described below.

When a user rotates the first handle 204 around the rotation shaft 243 while holding the first handle 204, the actuation manipulation part 203 and the yaw manipulation part 202 are yaw-rotated around the rotation shaft 243. That is, when the pulley 210 of the first actuation manipulation part 251 to which the wires 301 and 305 are fixedly coupled is rotated around the rotation shaft 243, the wires 301 and 305 wound around the pulleys 211 and 212 are moved. Similarly, when the pulley 220 of the second actuation manipulation part 256, to which the wires 302 and 306 are fixedly coupled, is rotated around the rotation shaft 243, the wires 302 and 306 wound around the pulleys 221 and 222 are moved. At this time, the wires 301 and 305 connected to the first jaw 101 and the wires 302 and 306 connected to the second jaw 102 are wound around the pulleys 211 and 212 and the pulleys 221 and 222, so that the first jaw 101 and the second jaw 102 are rotated in the same direction during the yaw motion. In addition, a rotating force is transmitted to the end tool 100 through the power transmission part 300, and thus a yaw motion in which two jaws 103 of the end tool 100 are rotated in the same direction is performed.

At this time, since the yaw frame 207 connects the first handle 204, the rotation shaft 241, the rotation shaft 242, and the rotation shaft 243 to each other, the first handle 204, the yaw manipulation part 202, and the actuation manipulation part 203 are rotated together around the rotation shaft 243.

Next, the pitch motion will be described below.

When a user rotates the first handle 204 around the rotation shaft 246 while holding the first handle 204, the actuation manipulation part 203, the yaw manipulation part 202, and the pitch manipulation part 201 are pitch-rotated around the rotation shaft 246. That is, when the pulley 210 of the first actuation manipulation part 251 to which the wires 301 and 305 are fixedly coupled is rotated around the rotation shaft 246, the wires 301 and 305 wound around the pulley 217 and the pulley 218 are moved. Similarly, when the pulley 220 of the second actuation manipulation part 256, to which the wires 302 and 306 are fixedly coupled, is rotated around the rotation shaft 246, the wires 302 and 306 wound around the pulley 227 and the pulley 228 are moved. At this time, as described with reference to FIG. 6, in order to allow the first jaw 101 and the second jaw 102 to pitch-rotate, the wires 301 and 305, which are first jaw wires, are moved in the same direction and respectively wound around the pulley 217 and the pulley 218, which are manipulation part pitch main pulleys, and the wires 302 and 306, which are second jaw wires, are moved in the same direction and respectively wound around the pulley 227 and the pulley 228, which are manipulation part pitch main pulleys. In addition, a rotating force is transmitted to the end tool 100 through the power transmission part 300, and two jaws 103 of the end tool 100 perform the pitch motion.

At this time, since the pitch frame 208 is connected to the yaw frame 207, and the yaw frame 207 connects the first handle 204, the rotation shaft 241, the rotation shaft 242, and the rotation shaft 243 to each other, when the pitch frame 208 is rotated around the rotation shaft 246, the yaw frame 207, the first handle 204, the rotation shaft 241, the rotation shaft 242, and the rotation shaft 243 connected to the pitch frame 208 are rotated together with the pitch frame 208. That is, when the pitch manipulation part 201 is rotated around the rotation shaft 246, the actuation manipulation part 203 and the yaw manipulation part 202 are rotated together with the pitch manipulation part 201.

In summary, in the surgical instrument 10 according to an embodiment of the present disclosure, the pulleys are formed on respective joint points (an actuation joint, a yaw joint, and a pitch joint), the wires (the first jaw wire or the second jaw wire) are wound around the pulleys, the rotational manipulations (actuation rotation, yaw rotation, and pitch rotation) of the manipulation part cause the movement of each wire, which in turn induces the desired motion of the end tool 100. Furthermore, the auxiliary pulley may be formed at one side of each of the pulleys, and the wire may not be wound several times around one pulley due to the auxiliary pulley.

FIG. 10 is a view schematically illustrating only a configuration of pulleys and wires constituting joints of the surgical instrument 10 according to an embodiment of the present disclosure illustrated in FIG. 2. In FIG. 10, the relay pulleys that are not related to the operation of joints and are used to reroute the wire are omitted.

Referring to FIG. 10, the manipulation part 200 may include the pulley 210, the pulley 211, the pulley 212, the pulley 213, the pulley 214, the pulley 215, the pulley 216, the pulley 217, and the pulley 218 that are related to a rotational motion of the first jaw 101.

In addition, the manipulation part 200 may include the pulley 220, the pulley 221, the pulley 222, the pulley 223, the pulley 224, the pulley 225, the pulley 226, the pulley 227, and the pulley 228 that are related to a rotational motion of the second jaw 102. (the arrangement and structure of each of the pulleys of the manipulation part 200 are the same in principle as the arrangement and structure of each of the pulleys of the end tool 100, and thus specific designations of some reference numerals are omitted in the drawings).

The pulleys 211 and 212 and the pulleys 221 and 222 may be formed to be rotatable independently of each other around the same shaft, that is the rotation shaft 243. In this case, the pulleys 211 and 212 and the pulleys 221 and 222 may each be formed as two pulleys formed to face each other and formed to be independently rotatable.

The pulleys 213 and 214 and the pulleys 223 and 224 may be formed to be rotatable independently of each other around the same shaft, that is the rotation shaft 244. Here, the pulleys 213 and 214 may be formed as two pulleys formed to face each other and formed to be independently rotatable, and in this case, the two pulleys may be formed to have different diameters. Similarly, the pulleys 223 and 224 may be formed as two pulleys formed to face each other and formed to be independently rotatable, and in this case, the two pulleys may be formed to have different diameters.

The pulleys 215 and 216 and the pulleys 225 and 226 may be formed to be rotatable independently of each other around the same shaft, that is the rotation shaft 245. In this case, the pulleys 215 and 216 may be formed to have different diameters. In addition, the pulleys 225 and 226 may be formed to have different diameters.

The pulleys 217 and 218 and the pulleys 227 and 228 may be formed to be rotatable independently of each other around the same shaft, that is the rotation shaft 246.

The wire 301 is wound around the pulley 210 after sequentially passing through the pulley 217, the pulley 215, the pulley 213, and the pulley 211 of the manipulation part 200, and then is coupled to the pulley 210 by the coupling member 324. Meanwhile, the wire 305 sequentially passes through the pulley 218, the pulley 216, the pulley 214, and the pulley 212 of the manipulation part 200 and is coupled to the pulley 210 by the coupling member 324. Thus, when the pulley 210 is rotated, the wires 301 and 305 are wound around or released from the pulley 210, and accordingly, the first jaw 101 is rotated.

The wire 306 is wound around the pulley 220 after sequentially passing through the pulley 227, the pulley 225, the pulley 223, and the pulley 221 of the manipulation part 200, and then is coupled to the pulley 220 by the coupling member 327. Meanwhile, the wire 302 sequentially passes through the pulley 228, the pulley 226, the pulley 224, and the pulley 222 of the manipulation part 200 and is coupled to the pulley 220 by the coupling member 327. Thus, when the pulley 220 is rotated, the wire 302 and the wire 306 are wound around or released from the pulley 220, and accordingly, the second jaw 102 is rotated.

### (Pitch-Related Pulley)

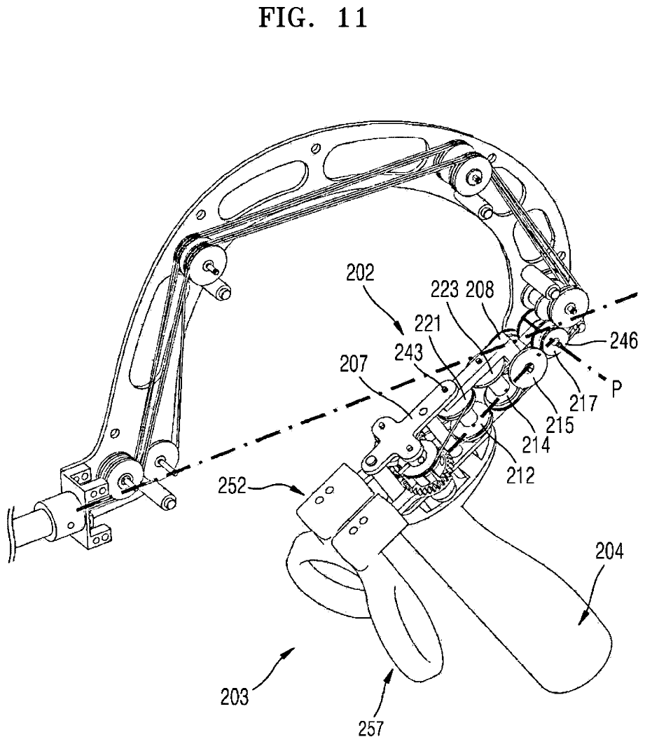

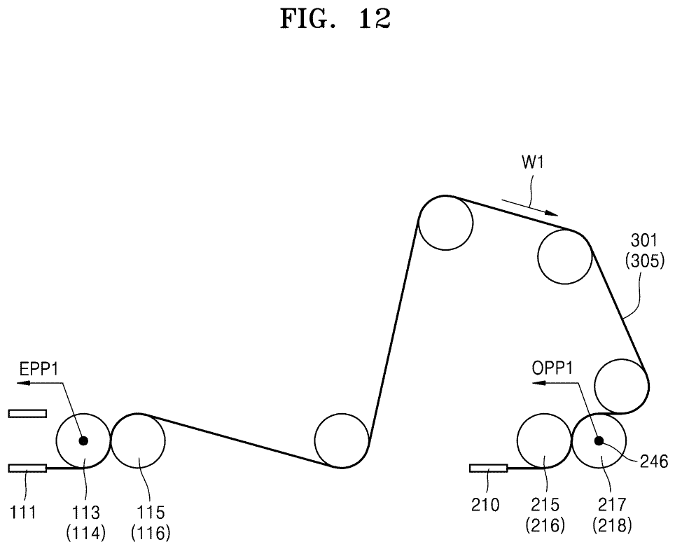

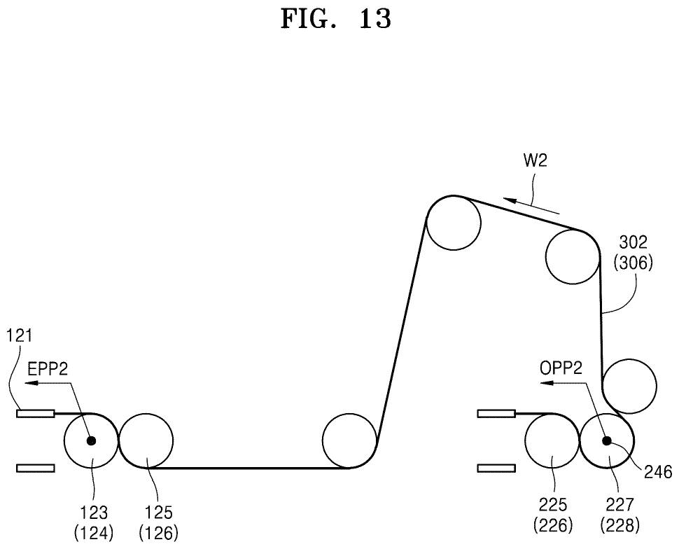

FIG. 11 is a perspective view illustrating a pitch motion of the surgical instrument of FIG. 2. FIGS. 12 and 13 are diagrams illustrating a configuration of pulleys and wires, which are related to a pitch motion of the surgical instrument 10 according to an embodiment of the present disclosure illustrated in FIG. 2, in detail for each of the first jaw and the second jaw. FIG. 12 is a diagram illustrating only pulleys and wires related to the first jaw, and FIG. 13 is a diagram illustrating only pulleys and wires related to the second jaw. As shown in FIG. 6 and elsewhere herein, there are two pulleys related to a pitch motion, and both strands of each wire are wound in the same path, which is illustrated with one line in FIGS. 12 and 13.

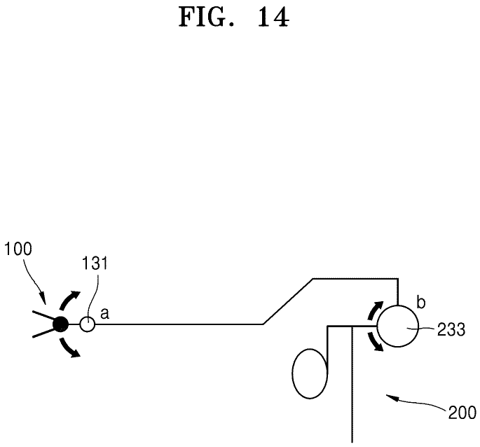

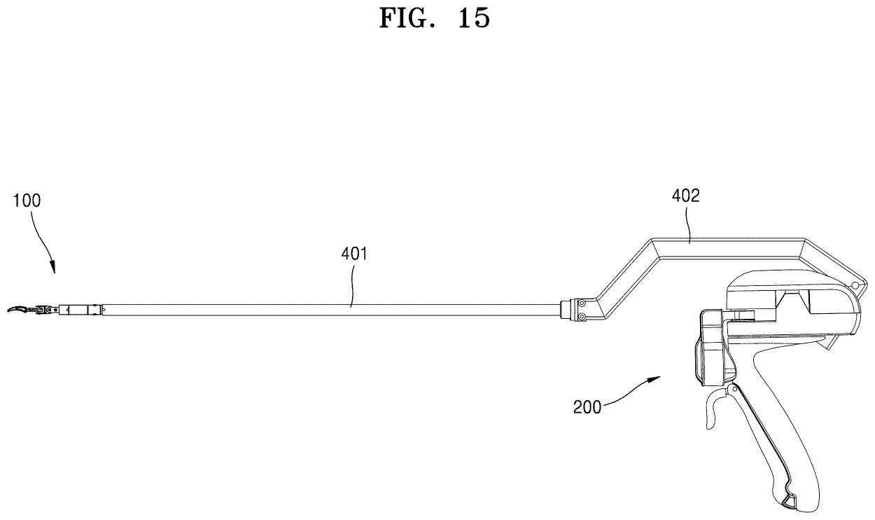

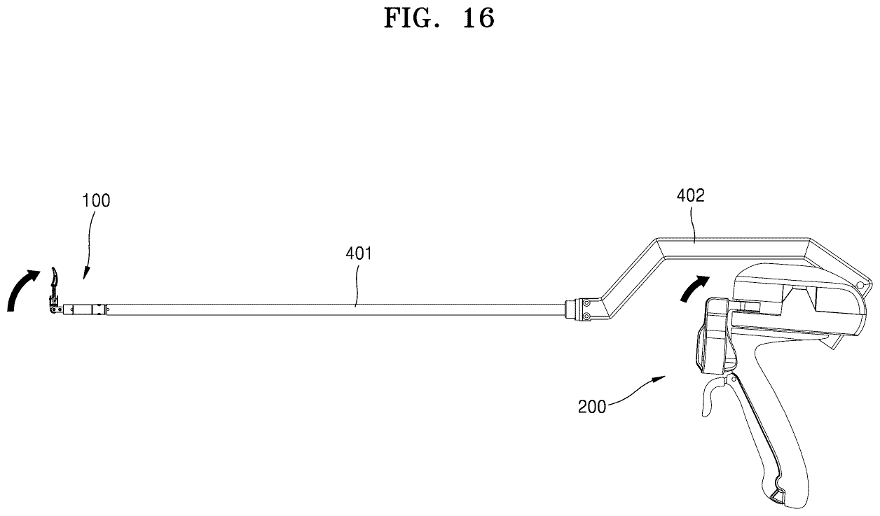

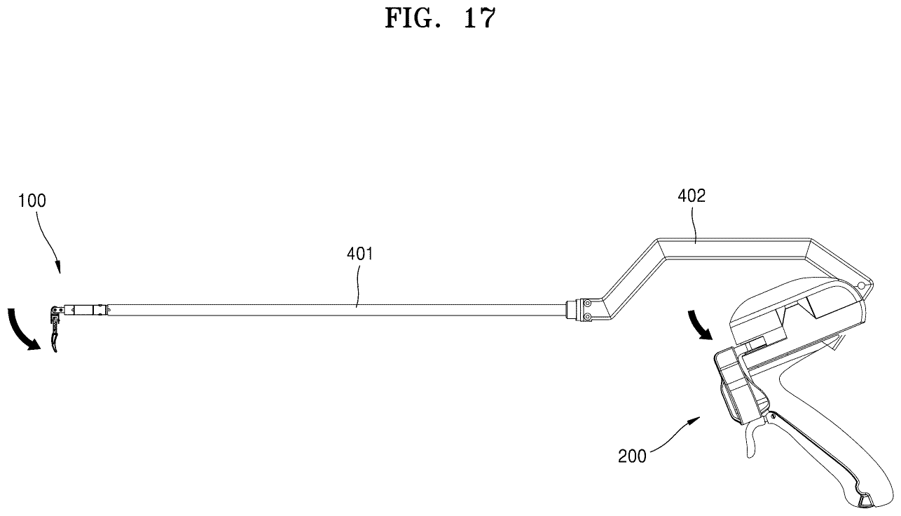

FIG. 14 is a conceptual diagram for describing a pitch motion in relation to a diameter ratio of the pitch pulleys of the surgical instrument illustrated in FIG. 2, and FIGS. 15 to 17 are side views illustrating a pitch motion of the surgical instrument according to the first embodiment of the present disclosure. Here, FIG. 15 is a view illustrating a state of the manipulation part when the end tool is parallel to a first direction, i.e., a pitch motion is not performed, FIG. 16 is a view illustrating a rotation radius of the manipulation part when the end tool is rotated by +90°, and FIG. 17 is a view illustrating a rotation radius of the manipulation part when the end tool is rotated by −90°.

Referring to FIG. 12, when the first handle 204 is rotated around the rotation shaft 246 in the direction of an arrow OPP1, the pulley 210, the pulley 215, the pulley 217, and the like, and the wire 301 and the like wound therearound are rotated as a whole around the rotation shaft 246. At this time, since the wires 301 and 305, which are first jaw wires, are wound around upper portions of the pulley 217 and the pulley 218 as shown in FIG. 12, the wires 301 and 305 are moved in the direction of an arrow W1. As a result, as described with reference to FIG. 5, the first jaw 101 of the end tool 100 is rotated in the direction of an arrow EPP1.

Referring to FIG. 13, when the first handle 204 is rotated around the rotation shaft 246 in the direction of an arrow OPP2, the pulley 220, the pulley 225, the pulley 227, and the like, and the wire 302 and the like wound therearound are rotated as a whole around the rotation shaft 246. At this time, since the wires 302 and 306, which are second jaw wires, are wound around lower portions of the pulley 227 and the pulley 228 as shown in FIG. 13, the wires 302 and 306 are moved in the direction of an arrow W2. As a result, as described with reference to FIG. 5, the second jaw 102 of the end tool 100 is rotated in the direction of an arrow EPP2.

Thus, the actuation, yaw, and pitch manipulations can be manipulated independent of each other.

As described with reference to FIG. 1, the actuation manipulation part 203, the yaw manipulation part 202, and the pitch manipulation part 201 are configured such that the respective rotation shafts are located at the rear thereof to be identical to the joint configuration of the end tool, so that a user may intuitively perform matching manipulations.

In particular, in the surgical instrument 10 according to an embodiment of the present disclosure, the pulleys are formed on respective joint points (an actuation joint, a yaw joint, and a pitch joint), the wires (the first jaw wire or the second jaw wire) are formed to be wound around the pulleys, the rotational manipulations (actuation rotation, yaw rotation, and pitch rotation) of the manipulation part cause the movement of each wire, which in turn induces the desired motion of the end tool 100. Furthermore, the auxiliary pulleys may be formed on one side of the respective pulleys, and these auxiliary pulleys may prevent the wire from being wound around one pulley multiple times, so that the wires wound around the pulley do not come into contact with each other, and paths of the wire being wound around the pulley and the wire being released from the pulley are safely formed, so that safety and efficiency in the transmission of driving force of a wire may be improved.

Meanwhile, as described above, the yaw manipulation part 202 and the actuation manipulation part 203 are directly formed on the first handle 204. Thus, when the first handle 204 is rotated around the rotation shaft 246, the yaw manipulation part 202 and the actuation manipulation part 203 are also rotated together with the first handle 204. Accordingly, the coordinate systems of the yaw manipulation part 202 and the actuation manipulation part 203 are not fixed, but are continuously changed relative to the rotation of the first handle 204. That is, in FIG. 2 or the like, the yaw manipulation part 202 and the actuation manipulation part 203 are illustrated as being parallel to the z-axis. However, when the first handle 204 is rotated, the yaw manipulation part 202 and the actuation manipulation part 203 are not parallel to the Z-axis any longer. That is, the coordinate systems of the yaw manipulation part 202 and the actuation manipulation part 203 are changed according to the rotation of the first handle 204. However, in the present specification, for convenience of description, unless described otherwise, the coordinate systems of the yaw manipulation part 202 and the actuation manipulation part 203 are described on the basis of a state in which the first handle 204 is located perpendicular to the connection part 400 as illustrated in FIG. 2.

### (Pitch-Related Pulley)

The pulleys of the manipulation part 200 have a one-to-one corresponding relationship with the pulleys of the end tool 100.

The pitch pulleys will be described first.

In detail, the pulley 113, the pulley 114, the pulley 123, and the pulley 124, which are rotated around the end tool pitch rotation shaft 143 to perform a pitch motion in the end tool 100, have a one-to-one corresponding relationship with the first jaw pitch pulley 217, the first jaw pitch pulley 218, the second jaw pitch pulley 227, and the second jaw pitch pulley 228, which are rotated around the pitch rotation shaft 246 to perform the pitch motion in the manipulation part 200.

Here, for convenience, the pulley 113, the pulley 114, the pulley 123, and the pulley 124 of the end tool 100 are grouped together and referred to as end tool jaw pitch main pulleys, and the first jaw pitch pulley 217, the first jaw pitch pulley 218, the second jaw pitch pulley 227, and the second jaw pitch pulley 228 of the manipulation part 200 are grouped together and referred to as manipulation part pitch main pulleys.

In summary, when the manipulation part pitch main pulley is rotated around the pitch rotation shaft 246, the end tool jaw pitch main pulley is rotated around the end tool pitch rotation shaft 143, thereby performing the pitch motion.

Here, in the present embodiment, a diameter of (at least some of) the end tool jaw pitch main pulleys and a diameter of (at least some of) the manipulation part pitch main pulleys are formed to be different from each other. In addition, since the diameter of (at least some of) the end tool jaw pitch main pulleys and the diameter of (at least some of) the manipulation part pitch main pulleys are formed to be different from each other as described above, a rotation angle of (at least some of) the end tool jaw pitch main pulleys a rotation angle of (at least some of) the manipulation part pitch main pulleys are different from each other. This will be described in more detail below.

First, the end tool 100 has to perform a motion ranging from at least +90° to −90° (or more) with respect to the Y-axis for the pitch motion. On the other hand, an operating range of the wrist of a user gripping the manipulation part 200 ranges from about at least +60° to −60° around the Y-axis (although there may be individual differences between people). Accordingly, when the rotation angle of (at least some of) the end tool jaw pitch main pulleys is the same as the rotation angle of (at least some of) the manipulation part pitch main pulleys, an operating range of the end tool 100 is restricted within the operating range of the wrist of the user, and thus the end tool 100 will not be operated at the required angle. Alternatively, when the user forcibly twists the wrist by 90° to operate the end tool 100, which increases user fatigue and strains the body, resulting in an ergonomically undesirable design.

Furthermore, in the surgical instrument according to the present disclosure, the bent part (see 402 in FIG. 3) formed on the connection part (see 400 in FIG. 3), and the shape of the bent part (see 402 in FIG. 3) may restrict the operating range of the manipulation part 200.

In addition, by forming (at least some of) the end tool jaw pitch main pulleys and (at least some of) the manipulation part pitch main pulleys, which are pitch driving pulleys corresponding to each other, to have different diameters, the rotation angle of (at least some of) the end tool jaw pitch main pulleys and the rotation angle of (at least some of) the manipulation part pitch main pulleys may be adjusted to be different from each other.

That is, when the manipulation part pitch main pulleys and the end tool jaw pitch main pulleys are rotated together, lengths of the wires wound around the manipulation part pitch main pulleys and the end tool jaw pitch main pulleys are equally changed. Thus, when the wires are moved by the same length, by adjusting the diameters of the pulleys around which the wires are wound, the rotation angles of the pulleys around which the wires are wound are made to be different from each other. Describing this with reference to FIG. 14, when a radius of an end tool pitch pulley a is ra and a radius of a manipulation part pitch pulley b is rb, a rotation ratio of the pulley a to the pulley b is rb:ra. That is, when the radius rb of the manipulation part pitch pulley b is greater than the radius ra of the end tool pitch pulley a, the end tool 100 may be largely rotated even with less rotation of the manipulation part 200.

In particular, since even with less rotation of the manipulation part pitch main pulley, the corresponding end tool jaw pitch main pulley must be rotated at an angle larger than that of the manipulation part pitch main pulley, the diameter of (at least some of) the end tool jaw pitch main pulleys may be formed smaller than the diameter of (at least some of) the manipulation part pitch main pulleys.

The following condition may be required:
1:1.5≤diameter of end tool jaw pitch main pulley:diameter of manipulation part pitch main pulley≤1:4.5
may be required. This will be described below in more detail.

As described above, a required operating range for the pitch motion of the end tool 100 ranges from +90° to −90°, while an operating range of the wrist of the user gripping the manipulation part 200 ranges from about +60° to −60°. Thus, when the ratio of the diameter of the end tool jaw pitch main pulley to the diameter of the manipulation part pitch main pulley is at least 1:1.5 more, the operating range of the user's wrist matches the required operating range of the end tool 100.

Meanwhile, when the end tool 100 is excessively rotated even when the user moves the manipulation part 200 slightly, it is difficult to precisely and accurately manipulate the end tool 100. Thus, the ratio of the diameter of the end tool jaw pitch main pulley to the diameter of the manipulation part pitch main pulley cannot be increased indefinitely. Usability evaluation revealed that achieving the precise manipulation desired by the user becomes somewhat difficult when the ratio of the diameter of the end tool jaw pitch main pulley to the diameter of the manipulation part pitch main pulley exceeds 1:4.5.

Thus, the relationship of 1:1.5≤diameter of end tool jaw pitch main pulley:diameter of manipulation part pitch main pulley≤1:4.5 may be required.

Meanwhile, as described above, in order to reduce the height of the bent part 402, i.e., the height of the arc-shaped frame, the amount of rotation of the manipulation part 200 should be reduced relative to the end tool 100. Thus, the ratio of the diameter of the end tool jaw pitch main pulley to the diameter of the manipulation part pitch main pulley needs to be changed to be greater than at least 1:1.5. In addition, in terms of structural design, when the ratio of the diameter of the end tool jaw pitch main pulley to the diameter of the manipulation part pitch main pulley is 1:3.5 or more, the height of the arc-shaped frame can be reduced by approximately 10 mm compared to the conventional structure.

Thus, preferably, the following condition may be required:
1:1.3.5≤diameter of end tool jaw pitch main pulley:diameter of manipulation part pitch main pulley≤1:4.5
may be required. This will be described in detail below.

For example, when the diameter of the end tool jaw pitch main pulley is 6 mm and the diameter of the manipulation part pitch main pulley is 15 mm, a rotation ratio of the manipulation part pitch main pulley to the manipulation part pitch main pulley may be considered to be 2.5:1. That is, when the manipulation part 200 is pitch-rotated by 36°, the end tool 100 is pitch-rotated by 90°. In addition, by further adjusting the diameter of the pulley, the amount of rotation of the manipulation part 200 may be reduced compared to the conventional manipulation part. For example, when the diameter of the end tool jaw pitch main pulley is 4.4 mm and the diameter of the manipulation part pitch main pulley is 17.6 mm, the rotation ratio of the end tool jaw pitch main pulley to the manipulation part pitch main pulley may be considered to be 4:1, and when rotating the manipulation part 200 by 22.5°, the end tool 100 may be rotated by 90°.

Accordingly, as shown in FIGS. 16 and 17, in the surgical instrument according to the first embodiment of the present disclosure, by adjusting the operating range of the end tool 100 to be between +90° and −90° while the operating range of the manipulation part 200 to be between +22.5° and −22.5° during the pitch motion, the height of the arc-shaped frame, which is the bent part 402, can be formed to be lower as the amount of pitch rotation of the manipulation part 200 is reduced.

In other words, whereas in the conventional surgical instrument, in order to pitch-rotate the end tool by +90°, the manipulation part 200 has to pitch-rotate by +36°, in the surgical instrument according to the first embodiment of the present disclosure, in order to pitch-rotate the end tool 100 by +90°, the manipulation part 200 only has to pitch-rotate by +22.5°, thus reducing the operating range of the manipulation part 200. Accordingly, the bent part 402 may be shaped to be low to the extent that the bent part 402 does not restrict the operating range of the manipulation part 200.

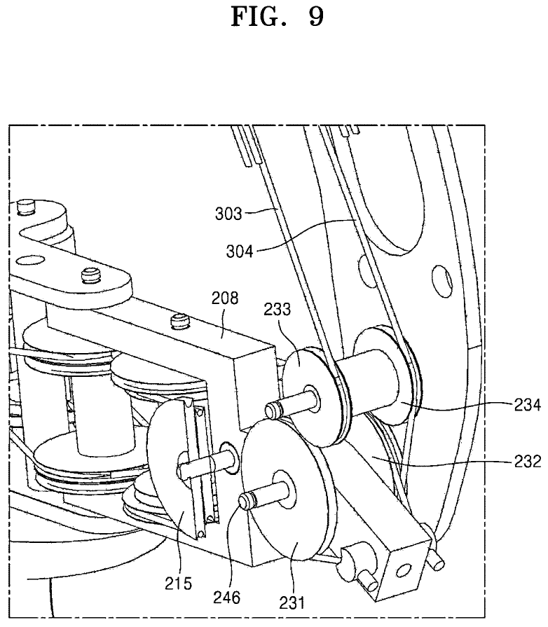

Meanwhile, although not shown in the drawings, the surgical instrument 10 according to the present embodiment may further include the end tool pitch pulley (see 131 in FIG. 5) formed on the end tool 100 side, the manipulation part pitch wire pulleys (see 231 and 232 in FIG. 9) formed on the manipulation part 200 side, and pitch wires (see 303 and 304 in FIG. 9) connecting the end tool pitch pulley to the manipulation part pitch wire pulleys. At this time, (diameter of end tool pitch pulley:diameter of manipulation part pitch wire pulley) may be formed to be equal to (diameter of end tool jaw pitch main pulley:diameter of manipulation part pitch main pulley).

The end tool pitch pulley 131 will be described first.

The end tool pitch pulley 131 may be formed on the end portion of the end tool hub 180 at the proximal end side. The end tool pitch pulley 131 may be formed integrally with the end tool hub 180 as one body, but the end tool pitch pulley 131 may be formed as a separate member from the end tool hub 180 and coupled to the end tool hub 180. Further, as shown in FIGS. 4 and 5, the end tool pitch pulley 131 may be formed to perform a pitch motion while rotating around the pitch rotation shaft 143. Meanwhile, the end tool pitch pulley 131 may be formed to have a size different from those of the pulley 113, the pulley 114, the pulley 123, and the pulley 124, which are pitch pulleys coupled to the same pitch rotation shaft 143.

The end tool pitch pulley 131 is coupled to one end portion of each of the wires 303 and 304, which are pitch wires, and is formed to operate together with the manipulation part pitch wire pulley 231, which is coupled to another end portion of each of the pitch wires located at the manipulation part 200 side, in response to the rotation of the manipulation part pitch wire pulley. The operation of the pitch wire will be described in detail below.

Referring to FIG. 9, the manipulation part pitch wire pulley 231 may formed to rotate together therewith the pitch frame 208 by being fixedly coupled thereto. In addition, the pitch wire 303 is fixedly coupled to the pitch frame 208 via the pitch wire auxiliary pulley 233 and the manipulation part pitch wire pulley 231. As a result, the pitch frame 208 and the manipulation part pitch wire pulley 231 may be rotated together around the pitch rotation shaft 246 by the pitch rotation of the manipulation part 200.

The operation of the pitch wires 303 and 304 is described in detail as follows.

The end tool pitch pulley 131 is formed in the end tool 100 to be fixedly coupled to the end tool hub 180, and the manipulation part pitch wire pulley 231 is formed in the manipulation part 200. The end tool pitch pulley 131 and the manipulation part pitch wire pulley 231 are connected to each other by the pitch wire 303 so that the pitch motion of the end tool may be easily performed in response to the pitch motion of the manipulation part 200. Here, the end of the pitch wire 303 is fixedly coupled to the pitch frame 208 via the corresponding pitch wire auxiliary pulley 233 and manipulation part pitch wire pulley 231, and the manipulation part pitch wire pulley 231 is also fixedly coupled to the pitch frame 208. Similarly, the end of the pitch wire 304 is fixedly coupled to the pitch frame 208 via the corresponding pitch wire auxiliary pulley 234 and manipulation part pitch wire pulley 232, and the manipulation part pitch wire pulley 232 is also fixedly coupled to the pitch frame 208. That is, the pitch frame 208 and the manipulation part pitch wire pulleys 231 and 232 are rotated together around the pitch rotation shaft 246 by the pitch rotation of the manipulation part. As a result, both sides the pitch wire 303 and the pitch wire 304 are moved in opposite directions to transmit additional pitch rotation power separately from the pitch motion of the end tool 100 by the first jaw wire and the second jaw wire.

In summary, when the manipulation part pitch wire pulley of the manipulation part is rotated around the pitch rotation shaft 246, the end tool pitch pulley 131 is rotated around the end tool pitch rotation shaft 143, thereby performing the pitch motion.

Here, in the present embodiment, the end tool pitch pulley 131 and the manipulation part pitch wire pulley are formed to have different diameters. In addition, by forming the end tool pitch pulley and the manipulation part pitch wire pulley to have different diameters as described above, the rotation angle of the end tool pitch pulley and the rotation angle of the manipulation part pitch wire pulley are different from each other.

That is, when the manipulation part pitch wire pulley and the end tool pitch pulley are rotated together, the lengths of the wires wound around these pulley are equally changed. Thus, when the wires are moved by the same length, by adjusting the diameters of the pulleys around which the wires are wound, the rotation angles of the pulleys around which the wires are wound are made to be different from each other.

This will be described in more detail below.

In particular, the end tool pitch pulley may be formed to have a diameter smaller than a diameter of the manipulation part pitch wire pulley because, even when the manipulation part pitch wire pulley rotates only slightly, the end tool pitch pulley corresponding to the manipulation part pitch wire pulley should be rotated at a larger angle.

Here, the relationship of 1:1.5≤diameter of end tool pitch pulley:diameter of manipulation part pitch wire pulley≤1:4.5 may be required. This will be described below in more detail.

As described above, a required operating range for the pitch motion of the end tool 100 ranges from +90° to −90°, while an operating range of the wrist of the user gripping the manipulation part 200 ranges from about +60° to −60°. Thus, when the ratio of the diameter of the end tool pitch pulley to the diameter of the manipulation part pitch wire pulley is at least 1:1.5 more, the operating range of the user's wrist matches the required operating range of the end tool 100.

Meanwhile, when the end tool 100 is excessively rotated even when the user moves the manipulation part 200 slightly, it is difficult to precisely and accurately manipulate the end tool 100. Thus, the ratio of the diameter of the end tool pitch pulley to the diameter of the manipulation part pitch wire pulley cannot be increased indefinitely. Usability evaluation revealed that achieving the precise manipulation desired by the user becomes somewhat difficult when the ratio of the diameter of the end tool pitch pulley to the diameter of the manipulation part pitch wire pulley exceeds 1:4.5.

The following condition may be required:
1:1.5≤diameter of end tool pitch pulley:diameter of manipulation part pitch wire pulley≤1:4.5
may be required.

Meanwhile, as described above, in order to reduce the height of the bent part 402, i.e., the height of the arc-shaped frame, the amount of rotation of the manipulation part 200 should be reduced relative to the end tool 100. Thus, the ratio of the diameter of the end tool pitch pulley to the diameter of the manipulation part pitch wire pulley needs to be changed to be greater than at least 1:1.5. In addition, in terms of structural design, when the ratio of the diameter of the end tool pitch pulley to the diameter of the manipulation part pitch wire pulley is 1:3.5 or more, the height of the arc-shaped frame can be reduced by approximately 10 mm compared to the conventional structure.

Thus, preferably, the following condition may be required:
1:3.5≤diameter of end tool pitch pulley:diameter of manipulation part pitch wire pulley≤1:4.5
may be required. This will be described in detail below.

For example, when the diameter of the end tool pitch pulley is 6 mm and the diameter of the manipulation part pitch wire pulley of the manipulation part is 15 mm, a rotation ratio of the end tool pitch pulley to the manipulation part pitch wire pulley may be considered to be 2.5:1. That is, when the manipulation part 200 is pitch-rotated by 36°, the end tool 100 is pitch-rotated by 90°. In addition, by further adjusting the diameter of the pulley, the amount of rotation of the manipulation part 200 may be reduced compared to the conventional manipulation part. For example, when the diameter of the end tool pitch pulley is 4.4 mm and the diameter of the manipulation part pitch wire pulley is 17.6 mm, the rotation ratio of the end tool pitch pulley to the manipulation part pitch wire pulley may be considered to be 4:1, and when rotating the manipulation part 200 by 22.5°, the end tool 100 may be rotated by 90°.

Accordingly, as shown in FIGS. 16 and 17, in the surgical instrument according to the first embodiment of the present disclosure, by adjusting the operating range of the end tool 100 to be between +90° and −90° while the operating range of the manipulation part 200 to be between +22.5° and −22.5° during the pitch motion, the height of the arc-shaped frame, which is the bent part 402, can be formed to be lower as the amount of pitch rotation of the manipulation part 200 is reduced.

In other words, whereas in the conventional surgical instrument, in order to pitch-rotate the end tool by +90°, the manipulation part 200 has to pitch-rotate by +36°, in the surgical instrument according to the first embodiment of the present disclosure, in order to pitch-rotate the end tool 100 by +90°, the manipulation part 200 only has to pitch-rotate by +22.5°, thus reducing the operating range of the manipulation part 200. Accordingly, the bent part 402 may be shaped to be low to the extent that the bent part 402 does not restrict the operating range of the manipulation part 200.

### <Second Embodiment of Surgical Instrument>

Hereinafter, a surgical instrument 20 according to a second embodiment of the present disclosure will be described. Here, the surgical instrument 20 according to the second embodiment of the present disclosure is different from the surgical instrument 10 according to the first embodiment of the present disclosure described above in that operational integrity or the like of the manipulation part and the end tool is changed.

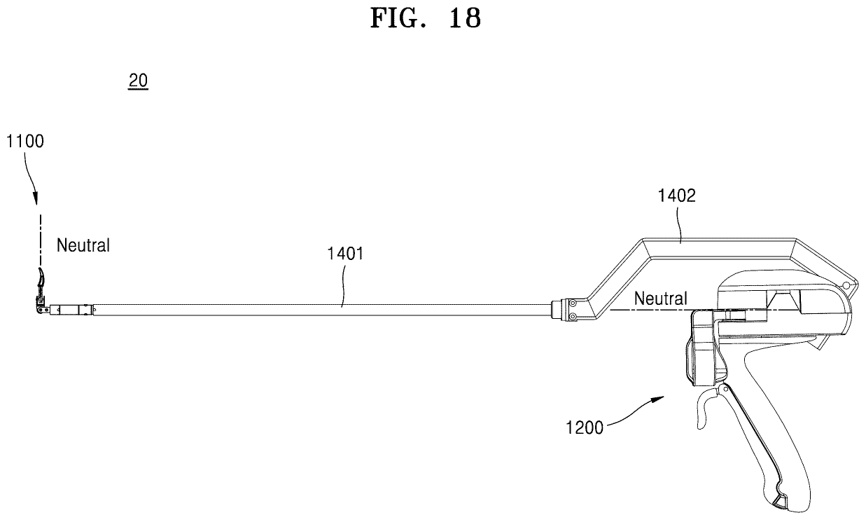

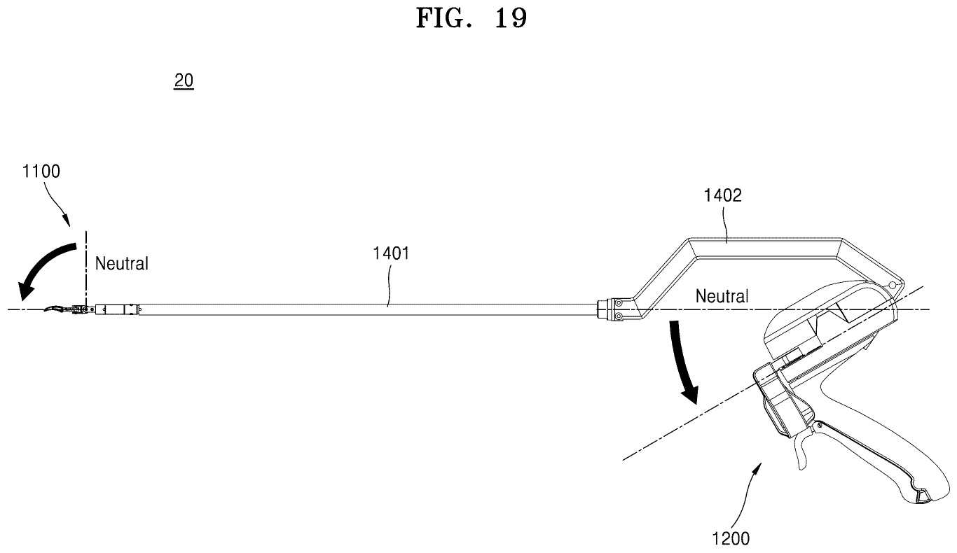

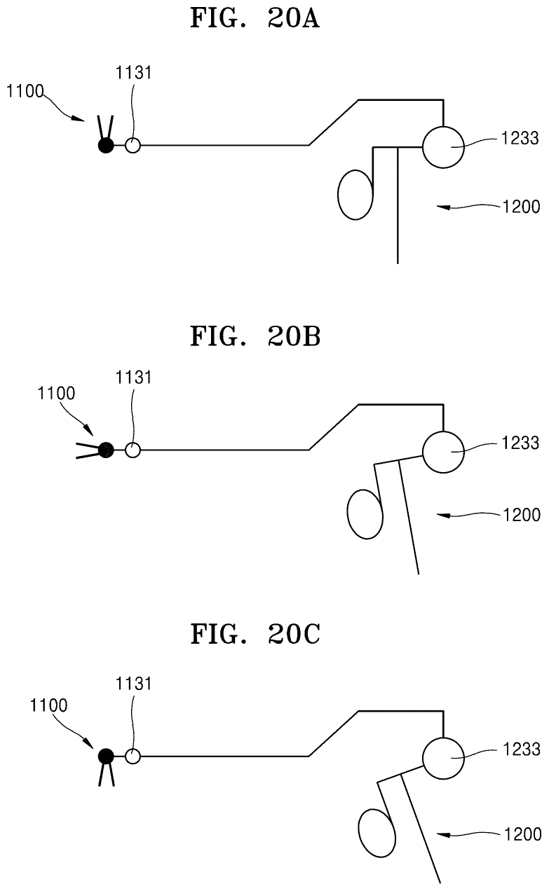

FIGS. 18 and 19 are side views illustrating a pitch motion of the surgical instrument according to the second embodiment of the present disclosure, and FIGS. 20A to 20C is a conceptual diagram illustrating the pitch motion of the surgical instrument of FIG. 17. Similar to that illustrated in FIG. 2, in the present drawings, a longitudinal direction of a connection part 1401 of the surgical instrument 20 according to the second embodiment of the present disclosure corresponds to the X-axis direction, a central axis of a pitch motion of a manipulation part 1200 corresponds to the Y-axis, and a plane one which a bent part 1402 is formed corresponds to the XZ plane. In the description of the present disclosure, for convenience, a case in which the bent part 1402 is formed above the XY plane, and the manipulation part 1200 is formed below the XY plane will be described by way of example. That is, the manipulation part 1200 is formed below the bent part 1402, and pitch rotating the manipulation part 1200 to be closer to the bent part 1402 will be described as rotating the manipulation part 1200 upward. However, the concept of the present disclosure is not limited thereto, and the surgical instrument may also be formed with the bent part below the manipulation part.

Referring to FIGS. 18 and 19, the surgical instrument 20 according to the second embodiment of the present disclosure may be in a state in which an end tool 1100 is pitch-rotated at a maximum rotation angle with respect to a first direction (X-axis) in a neutral state in which the manipulation part 1200 is parallel to the first direction. Specifically, when the manipulation part 1200 is in the neutral state, the end tool 1100 remains the +90° pitch-rotated state. In other words, when the end tool 1100 is pitch-rotated by +90°, the manipulation part 1200 may be in a non-rotated state, and when the end tool 1100 is parallel to the first direction, the manipulation part 1200 may be in a state of being rotated downward. In other words, it may be said that an angle of the end tool 1100 when the end tool 1100 is in a state parallel to the first direction does not match an angle of the manipulation part 1200 when the manipulation part 1200 is in the neutral state.

Meanwhile, referring to FIGS. 20A to 20C, as shown in FIG. 20A, since the end tool 1100 is in the +90° pitch-rotated state when the manipulation part 1200 is in the neutral state, the manipulation part 1200 needs to be rotated at a negative (−) angle to position the end tool 1100 parallel to the first direction. That is, as shown in FIG. 20B, the manipulation part 1200 is rotated further downward compared to when the manipulation part 1200 is in the neutral state. In addition, in order to rotate the end tool 1100 up to −90°, the manipulation part 1200 must be rotated to a greater angle than the state of the manipulation part 1200 in FIG. 20B.

Here, when a rotation ratio of an end tool jaw pitch main pulley to a manipulation part pitch main pulley is set as in the related art, a rotation angle range of the manipulation part required to rotate the end tool to 180° is increased, and particularly, when the manipulation part is controlled to pitch-rotate the end tool up to −90°, the user's wrist is bent by 90° or more, which does not secure user convenient, and thus, adjusting the rotation ratio of the pulley becomes necessary.

Thus, as described in the first embodiment, a diameter ratio of the end tool jaw pitch main pulley to the manipulation part pitch main pulley may be adjusted as follows.

That is, the relationship of 1:3.5≤diameter of end tool jaw pitch main pulley:diameter of manipulation part pitch main pulley≤1:4.5 may be required.

Likewise, as described in the first embodiment, a diameter ratio of an end tool pitch pulley to a manipulation part pitch wire pulley may be adjusted as follows.

That is, the relationship of 1:3.5≤diameter of end tool pitch pulley:diameter of manipulation part pitch wire pulley≤1:4.5 may be required.

Meanwhile, the +90° pitch-rotated state of the end tool 1100 when the manipulation part 1200 is in the neutral state as described above may implemented by setting an initial tension when the manipulation part 1200 is rotated to a negative (−) angle to correspond to when the end tool 1100 is in the state parallel to the first direction. That is, an initial position of the end tool may be adjusted by adjusting the length and tension of the wire coupled to the pitch pulley in the assembly operation.

Here, the case in which the manipulation part 1200 is in the neutral state, and the end tool 1100 is in the +90° pitch-rotated state is illustrated in the drawing. However, the concept of the present disclosure is not limited thereto, and in the state in which the end tool 1100 is parallel to the first direction, the manipulation part 1200 may be in a pitch-rotated state at a predetermined rotation angle with respect to the first direction. Specifically, it will be appreciated that there are various possible configurations of the manipulation part 1200 where the end tool 1100 is set to be parallel to the first direction when the manipulation part 1200 is in the pitch-rotated in the negative (−) direction. For example, in a case in which the end tool 1100 is in a state of being parallel to the first direction when the manipulation part 1200 is in a −10° pitch-rotated state, the end tool 1100 is in a −90° pitch-rotated state when the manipulation part 1200 is in a −30° pitch-rotated state, and the end tool 1100 is in a +90° pitch-rotated state when the manipulation part 1200 is in a +10° pitch-rotated state, the amount of rotation of the manipulation part 1200 in a positive (+) direction can be reduced, and thus the height of the bent part 1402 can be reduced accordingly. Accordingly, when a plurality of surgical instruments 20 are simultaneously used, interference between arc-shaped frames can be minimized.

### <Third Embodiment of Surgical Instrument>

Hereinafter, a surgical instrument 30 according to a third embodiment of the present disclosure will be described. Here, the surgical instrument 30 according to the third embodiment of the present disclosure is different from the surgical instrument 10 according to the first embodiment of the present disclosure described above in that the shape or the like of the bent part is changed.

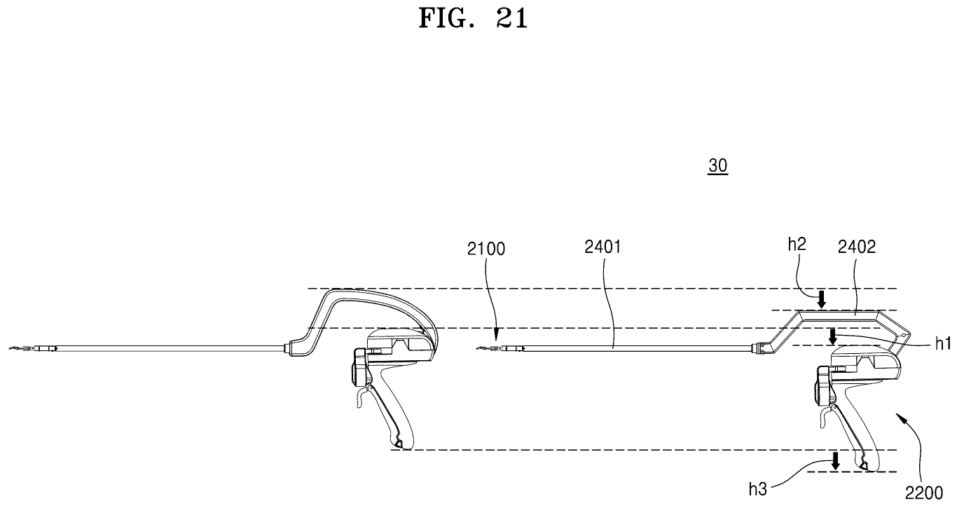

FIG. 21 is a side view illustrating a surgical instrument according to a third embodiment of the present disclosure.

Referring to FIG. 21, the surgical instrument 30 according to the third embodiment of the present disclosure includes an end tool 2100, a manipulation part 2200, a power transmission part (not shown), and a connection part 2400.

Here, the connection part 2400 is formed in the shape of a hollow shaft, and one or more wires and electric wires may be accommodated therein. The manipulation part 2200 is coupled to one end portion of the connection part 2400, the end tool 2100 is coupled to another end portion thereof, and the connection part 2400 may serve to connect the manipulation part 2200 to the end tool 2100.

Here, the connection part 2400 of the surgical instrument 30 according to the third embodiment of the present disclosure includes a straight part 2401 and a bent part 2402, the straight part 2401 is formed at a side of the connection part 2400 coupled to the end tool 2100, and the bent part 2402 is formed at a side of the connection part 2400, to which the manipulation part 2200 is coupled. As such, since the end portion of the connection part 2400 at the side of the manipulation part 2200 is formed to be bent, a pitch manipulation part 2201, a yaw manipulation part 2202, and an actuation manipulation part 2203 may be formed adjacent to an extension line of the end tool 2100. In other words, it may be said that the pitch manipulation part 2201 and the yaw manipulation part 2202 are at least partially accommodated in a concave portion formed by the bent part 2402. Due to the above-described shape of the bent part 2402, the shapes and motions of the manipulation part 2200 and the end tool 2100 may be further intuitively matched with each other. In the present embodiment, the end tool 2100 and the manipulation part 2200 are substantially the same as the end tool 100 and the manipulation part 200 described in the first embodiment, and thus detailed descriptions thereof will be omitted herein.

Meanwhile, as compared to the surgical instrument 10 according to the first embodiment, in the surgical instrument 30 according to the third embodiment of the present disclosure. the overall position of the manipulation part 2200 is lowered and the height of the bent part 2402, i.e., an arc-shaped frame, is correspondingly reduced.

In other words, in the surgical instrument 30 according to the third embodiment of the present disclosure, when the height of a lower end of the manipulation part 2200 is lowered by h3 and the height of an upper end of the manipulation part 2200 is lowered by h1 compared to the conventional surgical instrument, the height of the bent part 2402 may also be lowered by h2. In other words, when the size of the manipulation part 2200 of the surgical instrument 30 according to the third embodiment of the present disclosure is the same as the size of the manipulation part of the conventional surgical instrument, the height of the bent part 2402 may be eventually formed to be low by as much as the default position of the manipulation part 2200, which is set to be low. Accordingly, the height of the bent part 2402 can be lowered without restricting the operating range of the manipulation part 2200.

### <Fourth Embodiment of Surgical Instrument>

Hereinafter, a surgical instrument 40 according to a fourth embodiment of the present disclosure will be described. Here, the surgical instrument 40 according to the fourth embodiment of the present disclosure is different from the surgical instrument 10 according to the first embodiment of the present disclosure described above in that a pitch motion range or the like of each of the end tool and the manipulation part is changed.

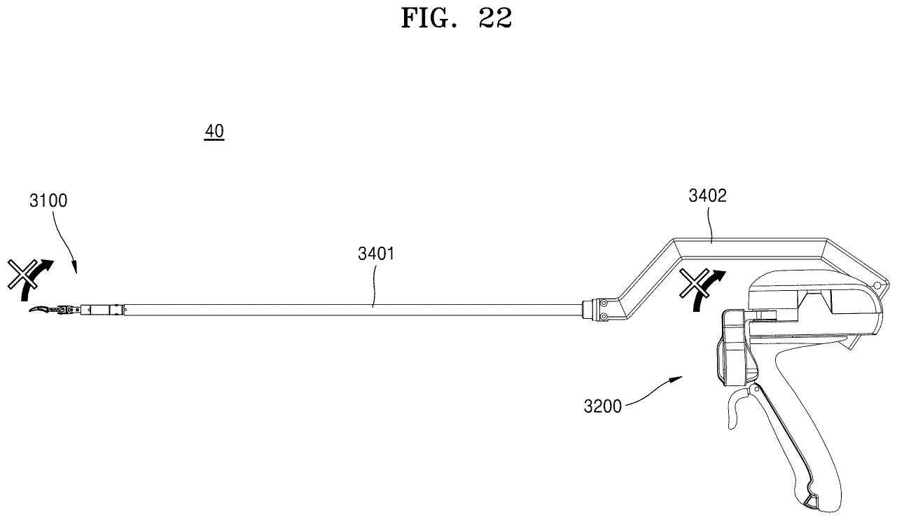

FIG. 22 is a side view illustrating the surgical instrument according to the fourth embodiment of the present disclosure.

Referring to FIG. 22, the surgical instrument 40 according to the fourth embodiment of the present disclosure includes an end tool 3100, a manipulation part 3200, a power transmission part (not shown), and a connection part 3400.

Here, the connection part 3400 of the surgical instrument 40 according to the fourth embodiment of the present disclosure includes a straight part 3401 and a bent part 3402, the straight part 3401 is formed at a side of the connection part 3400 coupled to the end tool 3100, and the bent part 3402 is formed at a side of the connection part 3400, to which the manipulation part 3200 is coupled.

In the present embodiment, the end tool 3100 and the manipulation part 3200 are substantially the same as the end tool 100 and the manipulation part 200 described in the first embodiment, and thus detailed descriptions thereof will be omitted herein.

Meanwhile, the surgical instrument 40 according to the fourth embodiment of the present disclosure may restrict an operating range of the manipulation part 3200 in order to lower the height of the bent part 3402, that is, an arc-shaped frame. For example, in a pitch motion of the end tool 3100, in order to pitch-rotate the end tool 3100 by +90°, the manipulation part 3200 is also pitch-rotated in a positive (+) direction on the Z-axis. Here, it is possible to prevent interference between the manipulation part 3200 and the bent part 3402 by restricting the pitch rotation of the manipulation part 3200 in the positive (+) direction. That is, in the present embodiment, the height of the bent part 3402 can be lowered by restricting a pitch motion range of the end tool 3100 and a pitch motion range of the manipulation part 3200, thereby minimizing interference between the arc-shaped frames when a plurality of surgical instruments 40 are simultaneously used.

The present disclosure has been described above in relation to its preferred embodiments. It will be understood by those skilled in the art that various changes in form and details may be made therein without departing from the essential features of the present disclosure. Therefore, the disclosed embodiments should be considered in descriptive sense only and not for purposes of limitation. The scope of the present disclosure is defined not by the detailed description of the disclosure but by the appended claims, and all differences within the scope will be construed as being included in the present disclosure.

According to the present disclosure, a surgical instrument according to embodiments of the present disclosure can minimize interference between frames even when a plurality of surgical instruments are in use, thereby increasing an instrument's operation radius and improving convenience and surgery speed for the surgical operator.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A surgical instrument comprising:
an end tool including one or more jaws formed to be rotatable, and an end tool jaw pitch main pulley formed to be rotatable around an end tool pitch rotation shaft;
a manipulation part configured to control a rotation of the end tool;
a power transmission part including one or more jaw wires that are connected to the manipulation part to transmit a rotation of the manipulation part to the one or more jaws; and
a connection part formed to extend in a first direction (an X-axis), having one end portion to which the end tool is coupled and another end portion to which the manipulation part is coupled to connect the end tool to the manipulation part, and including a bent part formed to be bent at least once while connecting the end tool to the manipulation part,
wherein the manipulation part includes a pitch manipulation part including a manipulation part pitch main pulley formed to be rotatable around a pitch rotation shaft, and configured to control a pitch motion of the end tool, and
a diameter of the manipulation part pitch main pulley is greater than a diameter of the end tool jaw pitch main pulley.

2. The surgical instrument of claim 1, wherein the bent part is bent on a plane perpendicular to the pitch rotation shaft of the pitch manipulation part.

3. The surgical instrument of claim 1, wherein a ratio of the diameter of the end tool jaw pitch main pulley to the diameter of the manipulation part pitch main pulley ranges from 1:3.5 to 1:4.5.

4. The surgical instrument of claim 1, wherein
the end tool jaw pitch main pulley is connected to the manipulation part pitch main pulley through the one or more jaw wires, and
a rotation angle of the end tool jaw pitch main pulley when the at least one or more jaw wires move is greater than a rotation angle of the manipulation part pitch main pulley when the at least one or more jaw wires move.

5. The surgical instrument of claim 4, wherein a ratio of the rotation angle of the manipulation part pitch main pulley to the rotation angle of the end tool jaw pitch main pulley ranges from 1:3.5 to 1:4.5.

6. The surgical instrument of claim 5, wherein the bent part is formed not to make contact with the manipulation part but to be positioned maximally adjacent to a rotation radius of the manipulation part, when the manipulation part is controlled to pitch-rotate the end tool at a maximum rotation angle.

7. The surgical instrument of claim 1, wherein
the end tool further includes an end tool hub formed to internally accommodate at least some of the one or more jaws, and an end tool pitch pulley formed on an end portion of the end tool hub at a proximal end side,
the power transmission part further includes a pitch wire coupled to the end tool pitch pulley to rotate the end tool pitch pulley,
the manipulation part further includes a manipulation part pitch wire pulley around which the pitch wire is wound, and
a diameter of the manipulation part pitch wire pulley is greater than a diameter of the end tool pitch pulley.

8. The surgical instrument of claim 7, wherein a ratio of the diameter of the end tool pitch pulley to the diameter of the manipulation part pitch wire pulley ranges from 1:3.5 to 1:4.5.

9. The surgical instrument of claim 7, wherein
the end tool pitch pulley is connected to the manipulation part pitch wire pulley through the pitch wire, and
a rotation angle of the end tool pitch pulley when the pitch wire moves is greater than a rotation angle of the manipulation part pitch wire pulley when the pitch wire moves.

10. The surgical instrument of claim 9, wherein a ratio of the rotation angle of the manipulation part pitch wire pulley to the rotation angle of the end tool pitch pulley ranges from 1:3.5 to 1:4.5.

11. A surgical instrument comprising:
an end tool including one or more jaws formed to be rotatable;
a manipulation part including a pitch manipulation part configured to control a pitch motion of the end tool, and configured to control a rotation of the end tool;
a power transmission part including one or more jaw wires that are connected to the manipulation part to transmit a rotation of the manipulation part to the one or more jaws; and
a connection part formed to extend in a first direction (an X-axis), having one end portion to which the end tool is coupled and another end portion to which the manipulation part is coupled to connect the end tool to the manipulation part, and including a bent part formed to be bent at least once while connecting the end tool to the manipulation part,
wherein, in a state in which the end tool is parallel to the first direction, the manipulation part is in a pitch-rotated state at a predetermined rotation angle with respect to the first direction.

12. The surgical instrument of claim 11, wherein in a neutral state in which the manipulation part is parallel to the first direction, the end tool is in a pitch-rotated state at a maximum rotation angle with respect to the first direction.

13. The surgical instrument of claim 11, wherein
the end tool further includes an end tool hub formed to internally accommodate at least some of the one or more jaws, and an end tool pitch pulley formed on an end portion of the end tool hub at a proximal end side,
the power transmission part further includes a pitch wire coupled to the end tool pitch pulley to rotate the end tool pitch pulley,
the manipulation part further includes a manipulation part pitch wire pulley around which the pitch wire is wound, and
a diameter of the manipulation part pitch wire pulley is greater than a diameter of the end tool pitch pulley.

14. The surgical instrument of claim 13, wherein a ratio of the diameter of the end tool pitch pulley to the diameter of the manipulation part pitch wire pulley ranges from 1:3.5 to 1:4.5.

15. The surgical instrument of claim 13, wherein
the end tool pitch pulley is connected to the manipulation part pitch wire pulley through the pitch wire, and
a rotation angle of the end tool pitch pulley when the pitch wire moves is greater than a rotation angle of the manipulation part pitch wire pulley when the pitch wire moves.

16. The surgical instrument of claim 15, wherein a ratio of the rotation angle of the manipulation part pitch wire pulley to the rotation angle of the end tool pitch pulley ranges from 1:3.5 to 1:4.5.
